(19) United States
(12) Patent Application Publication
(10) Pub. No.: US 2026/0166171 A1
(43) Pub. Date: Jun. 18, 2026

(54) DEVICES AND METHODS FOR EXTRACORPOREAL CELL TREATMENT

(71) Applicant: LUPAGEN, INC., Dallas, TX (US)

(72) Inventors: David Peritt, Skokie, IL (US); Nripendra Das, Irving, TX (US)

(21) Appl. No.: 19/126,228

(22) PCT Filed: Nov. 1, 2023
(86) PCT No.: PCT/US2023/036619

§ 371 Date: Apr. 30, 2025

Related U.S. Application Data
(60) Provisional application No. 63/421,342, filed on Nov. 1, 2022.

(51) Int. Cl. A61K 47/69; A61K 31/7105; A61K 31/711; A61K 38/02; A61K 45/06

(52) U.S. Cl. CPC A61K 47/6901; A61K 31/7105; A61K 31/711; A61K 38/02; A61K 45/06; A61K 47/6931

(57) ABSTRACT

The present disclosure features a method of preparing a population of payload-associated cell complexes (PACCs), as well as related methods of use thereof.

### CLAIM OF PRIORITY

The present application claims priority to U.S. Provisional Patent Application No. 63/421,342, filed on Nov. 1, 2022. The entire contents of the foregoing application are incorporated herein by reference in their entirety.

### BACKGROUND

Methods for introducing a payload into a desired cell type derived from a patient are useful in a number of biomedical fields. While a variety of ex vivo methods have been developed for the introduction of payloads such as nucleic acids, proteins or drugs into peripheral blood derived mononuclear cells (PBMCs), the cost, complexity and safety of these methods limit broad access and use in all but most serious diseases. Recent efforts to address these issues involve introduction of the payload and creation of therapeutic cells in the patient. However, these methods must contend with many challenges including delivery of the payload, biodistribution, off target impact, accurate dosing, immunogenicity to the delivery vehicle and limitations in ability to redose. As such, there is a need for new devices and methods to improve delivery of payloads to peripheral blood derived cells.

### SUMMARY

The present disclosure features a method of preparing a population of payload-associated cell complexes (PACCs), as well as related methods of use thereof. In an embodiment, a PACC comprises a cell in contact with a payload (e.g., a therapeutic agent, e.g., a nucleic acid or protein), wherein under certain conditions, the payload does not enter into the cell it is in contact with. In an embodiment, the cell is in direct contact with the payload. In an embodiment, the cell is indirectly in contact with the payload. In an embodiment, the cell is selected from a B cell, T cell, effector or regulatory T cell, hematopoietic stem cell (HSCs), natural killer cell, NK T cell, γ/δ T cell, and plasma cell. In an embodiment, the PACCs are introduced into the subject using a patient-connected closed-loop device.

In another aspect, the present disclosure features a method of providing a subject with a population of payload-associated cell complexes (PACCs), comprising providing a population of cells, e.g., a population of cells from the subject, wherein a cell in the population of cells comprises a binding target. In an embodiment, the method further comprises extracorporeally contacting the population of cells with a payload under conditions (e.g., time, temperature) sufficient for association of the payload with the cell comprising the binding target, wherein the conditions are not sufficient for entry of the payload into the cell with which it is associated, thus forming a population of PACCs. In another embodiment, the method further comprises introducing the population of PACCs into the subject. In an embodiment, the method compri

[symbol]

In an embodiment, the population of PACCs comprises at least 2, 3, 4, 5, 10, 25, 50, 75, 100, 250, 500, 750, 1,000, 5,000, 7,500, 10,000, 25,000, 50,000, 75,000, 100,000, 250,000, 500,000, or more PACCs. In an embodiment, the population of PACCs comprises at least 1 million or more PACCs, 5 million or more PACCs, 10 million or more PACCs, 50 million or more PACCs, or 100 million or more PACCs. In an embodiment, the percentage of cells associated with the payload in the population of PACCs is between 30%-90% total cells, e.g., 50%-80% total cells in the population. In an embodiment, the conditions sufficient for association of the payload with a cell within the population comprise contacting the cells with the payload for between about 0 to about 10 hours, e.g., about 1 hour to about 9 hours, about 2 hours to about 8 hours. In an embodiment, the payload is disposed in a delivery vehicle, e.g., a lipid nanoparticle, viral vector, vesicle, or a liposome. In an embodiment, the concentration of the payload delivery vehicle is higher than the concentration of cells in the patient sample. In another embodiment, the concentration of the payload delivery vehicle is lower than the concentration of cells in the patient sample.

In another aspect, the present disclosure features a method of forming in a subject a cell that is provided with a payload, the method comprising introducing a population of extracorporeally formed PACCs into the subject under conditions sufficient for transformation or transfection of the cell of the PACC with the payload of the PACC in the subject; and allowing the transformation/transfection. In an embodiment, the method further comprises a) connecting a parenteral inlet to the subject, wherein the parenteral inlet is adapted to parenterally receive blood from the subject; b) permitting the blood, or a fraction thereof, from the subject to pass through the parenteral inlet to an extracorporeal cell binding (ECCB) module configured to allow extracorporeal formation of a PACC; c) maintaining conditions in the ECCB module such that cells from the subject's blood and a payload form a PACC; and d) delivering the PACC to the subject via a parenteral outlet adapted to parenterally administer PACC to the subject.

In another aspect, the present disclosure provides a method of making a PACC, using methods described herein under conditions sufficient to prevent uptake or entry of the payload into the cells of the PACC.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limited.

### BRIEF DESCRIPTION OF THE FIGURES

The skilled person in the art will understand that the drawings described below are for illustration purposes only.

FIG. 1 is a schematic representing the general workflow of an exemplary system, combining an apheresis module with a binding module into a single extracorporeal closed-looped system.

FIG. 2 is a schematic of an exemplary binding chamber module containing an agitation tray. The schematic illustrates how the agitation tray moves to gently rock the sample.

FIGS. 3A-3D are a set of graphs illustrating binding of lipid nanoparticles to peripheral blood mononuclear cells (PBMCs) to form payload-associated cell complexes (PACCs). FIG. 3A is a graph demonstrating percent binding of LNP to T cells at 4° C. or at 37° C. FIG. 3B illustrates that LNP binding can be directed to a specific subset of cells (e.g., T cells) using a targeting moiety (e.g., an anti-CD3 antibody). FIG. 3C is a graph illustrating uptake (endoocytosis) of LNPs comprising DiD by T cells at 4° C. or at 37° C. after 0, 2, 4, 8, or 24 hours. FIG. 3D illustrates that T cell viability is not affected by CD3-targeted LNPs. Abbreviations: MFI: mean fluorescence intensity; DiD: DiIC18(5); 1,1′-dioctadecyl-3,3,3′,3′-tetramethylindodicarbocyanine, 4-chlorobenzenesulfonate salt.

FIGS. 4A-4B are graphs demonstrating binding to various subsets of PBMCs using either CD3-LNPs (FIG. 4A) or LNPs comprising cationic lipids (FIG. 4B).

FIGS. 5A-5C demonstrate binding of different LNPs to T cells at various temperatures. FIG. 5A is a schematic illustrating the difference between CD3-LNPs and CD3-LNPs comprising polyethylene glycol (PEG). FIG. 5B is a graph demonstrating binding of either CD3-LNPs or CD3-LNPs PEG at room temperature to T cells. FIG. 5C is a graph demonstrating binding of either CD3-LNPs or CD3-LNPs comprising PEG at room temperature to T cells.

FIG. 6A-6F are a set of illustrations and graphs demonstrating the binding and endocytosis of polymeric nanoparticles by T cells. In these illustrations, trypan blue was used to quench signal from externally bound polymeric nanoparticles. FIGS. 6A-6D are illustrations summarizing the experiment and the use of trypan blue. FIG. 6A demonstrates the binding and endocytosis of polymeric nanoparticles by T cells at 4° C. without trypan-blue. FIG. 6B demonstrates the binding and endocytosis of polymeric nanoparticles by T cells at 4° C. with trypan-blue. FIG. 6C demonstrates the binding and endocytosis of polymeric nanoparticles by T cells at 37° C. without trypan-blue. FIG. 6D demonstrates the binding and endocytosis of polymeric nanoparticles by T cells at 37° C. with trypan-blue. FIG. 6E is a graph illustrating the binding of polymeric nanoparticles of varying compositions at 4° C. FIG. 6F is a graph illustrating binding of polymeric nanoparticles of varying compositions at 37° C. Abbreviations: CTR=control.

FIG. 7 is a graph illustrating the relative transfection of DNA encoding green fluorescent protein (GFP) in Jurkat cells after 24 or 48 hours.

FIG. 8 is a graph demonstrating luciferase activity following incubation of Jurkat cells with polymeric nanoparticles of varying composition and cell to nanoparticle ratios.

### DETAILED DESCRIPTION

The present disclosure features devices and methods for the delivery of a payload to a cell extracorporeally to provide a population of payload-associated cell complexes (PACCs), useful in the prevention or treatment of a disease or disorder in a subject. The devices described herein provide for a method of transporting a population of cells, e.g., peripheral blood mononuclear cells (PBMCs), outside of a subject for a short time, contacting these cells with a payload (e.g., a payload enclosed within a delivery vehicle or associated with a delivery vehicle) to establish binding of the cells with the payload forming PACCs, and then delivery of the PACCs back to the subject. In an embodiment, the subject is connected to the device throughout the entire process. The methods described herein may be carried out in conditions sufficient to allow for binding of the payload with the subject-derived cells, but not to allow for entry of the payload into the cells. These methods provide many benefits over current ex vivo methods, including cost, speed, and simplicity by enabling in vivo payload delivery and activity. In addition, delivery of a payload to a cell extracorporeally to form PACCs has many benefits over non-extracorporeal in vivo methods as the device and methods described herein may increase the control of dose, reduce residual free particles, limit off target impact and lower immunogenicity to the vehicle further enabling redosing, potentially improving safety and efficacy profile and enhancing the modes of therapeutic delivery which are not possible in vivo.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

The articles “a” and “an” are used herein to refer to one or to more than one (e.g., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the terms “bead”, “microparticle”, “nanoparticle”, or “particle” is a structure of any shape and of any composition that is manipulatable or behaves according to predetermined principles and can act as a vehicle for payload delivery. Such beads, microparticles, nanoparticles, or particles can be comprised of any suitable material, such as glass or ceramics, and/or one or more polymers, such as, for example, nylon, polytetrafluoroethylene, polystyrene, polyacrylamide, sepharose, agarose, cellulose, cellulose derivatives, agarose or dextran, a metal, a metal alloy (e.g., FeO), lipid, protein, polymer, lipopeptide, or other additional materials. In an embodiment, the particle is a lipid nanoparticle. In an embodiment, the particle comprises cationic lipids. In another embodiment, the particle comprises a targeting moiety (e.g., a ligand or an antibody targeting a cell-specific protein).

As used herein, “closed-loop” refers to a system that is continuous, in which the contents of the system are connected to the subject for the duration of the method performed. For example, in the closed-loop system described herein, the population of cells derived from the subject is kept in the system for the duration of the method, and can be associated with a payload extracorporeally then re-introduced to the subject, e.g., without the subject being moved off-line or disconnected. In an embodiment, the closed-loop systems described herein may allow for introduction of exogenous materials, such as buffers and payload, but the subject-derived cells are not removed from the system and manipulated externally before being returned to the subject. In an embodiment, the system may further be “patient-connected,” in which the system is connected to the patient for a duration of the process (e.g., the entire process).

As used herein, “continuous flow” refers to the flow of blood from a subject to the modular bedside system and back to the subject in which non-target cells (e.g red blood cells) generally return to the subject whereas target cells can be adapted by an association binding module in the device and then returned to the subject; all in a closed-loop, subject-connected manner and in real-time. The term “continuous flow” does not exclude operations within a closed-loop system that occur while a cell population or sub-population is shunted or deposited for some period of time, into a holding, or incubating chamber or receptacle within the system nor does it preclude small amounts of cells removed offline for sampling and analytical testing.

As used herein, the term “extracorporeal” refers to outside of the subject. For example, the devices and methods described herein allow for associating a payload with subject-derived cells extracorporeally, or outside the subject. The extracorporeal devices and methods herein may form a closed-loop with the subject. In an embodiment, in the extracorporeal environment, blood flows from the subject to the extracorporeal system and back to the subject in a closed-loop. In an embodiment, in the methods described herein, in which non-target cells generally return to the subject without modification, whereas target cells are bound with or by the payload in the extracorporeal environment and then returned to the subject as PACCs.

As used herein, the term “payload” is any agent that capable of being delivered to a subject-derived cell. Payloads may include nucleic acids, peptides, proteins, lipids, small molecules, or any combination thereof. Exemplary payloads include DNA, RNA (e.g., mRNAs, modified mRNAs (mmRNAs), small interfering RNA (siRNA), micro RNA (miRNA)), oligonucleotides (e.g., antisense oligonucleotides), ribozymes, mini-circles, plasmids, immune-stimulating nucleic acids, antisense RNAs, antagomir, antimir, supermir, mini-circles, plasmids, vesicle (e.g., extracellular vesicles (EVs) and exosomes), small molecules, drug formulations, proteins, amino acids, DNA editing systems, activation receptors, or other material. Payloads may be formulated in a manner such that they can enter and transform, position, or otherwise reproducibly integrate DNA, RNA, mRNA siRNA, shRNA, mini-circles, plasmids, vesicles, EVs, exosomes, small molecules, drug formulations, proteins, amino acids, peptides, DNA editing systems, activation receptors, or other material in a target subject-derived cell. In some embodiments, payload is provided encapsulated or positioned in a vehicle as defined herein. In some embodiments, the payload is provided encapsulated or positioned in a vehicle and the vehicle is adapted to specifically bind or otherwise associate with a predetermined target cell. In some embodiments, the payload is provided encapsulated or positioned in a vehicle and the vehicle is adapted to specifically bind or otherwise associate with a predetermined target cell through the presence of a targeting molecule on the vehicle.

A “payload-associated cell complex” (PACC), as used herein, comprises a cell and a payload, wherein the payload and the cell are incubated together extracorporeally with sufficient proximity or affinity for one another (e.g., prior to introduction into a subject) and in conditions such that, when the payload-associated cell is introduced into a subject, the payload and the cell with which it is associated such that the payload can enter the cell, e.g., traverse the cell membrane or enter the cell through a vesicle-mediated delivery mechanism. In an embodiment, the payload is associated with the cell, e.g., through an interaction with the cell membrane or an entity associated with the cell membrane (e.g., ionic charge, hydrophobicity, a peptide, polypeptide, or lipid, e.g., an antibody or receptor). In an embodiment, the payload is covalently associated with the cell, e.g., through a covalent linkage to the cell membrane or an entity associated with the cell membrane (e.g., a protein or lipid, e.g., an antibody or receptor). In another embodiment, the payload is associated with the cell through ligand binding to a target on the cell (e.g., an antibody binding to a cell-specific target). For clarity, a PACC does not include a cell into which the payload has already entered, e.g., extracorporeally. In an embodiment, the payload is disposed on or within a vehicle, such as a lipid or polymeric nanoparticle, extracellular vesicles, exosome, or a viral vector.

A “subject” or “patient,” as these terms are used herein, is a human. In embodiments, the subject is a vertebrate animal (e.g., mammal, bird, fish, reptile, or amphibian). The subject may be a male or female of any age group, e.g., a pediatric subject (e.g., infant, child, adolescent) or adult subject (e.g., young adult, middle-aged adult, or senior adult)). The subject may have a disease or disorder, such as cancer. The terms subject and patient are used interchangeably.

As used herein, the term “vehicle” or a “payload delivery vehicle” is refers to a payload carrier adapted to encapsulate, hold or otherwise at least partially contain the payload. Generally, a vehicle is adapted or formulated in a manner that it is capable of transporting the payload in the extracorporeal environment and/or the in vivo environment. Generally, the vehicle is formed of, contains or comprises, for example, a liposome, a virus (including, without limitation, viral components, viral particles, polypeptide sequences comprising a virus or component or particle thereof, and/or nucleic acids encoding a virus or component or particle thereof), polymer (including, without limitation, polymer-based or containing particles or nanoparticles), inorganic, lipid (including, without limitation, lipid-based or containing micelles, particles or nanoparticles), a hybrid lipid-polymer nanoparticle, an EV, an exosome, a dendrimer, a metal nanoparticle, a nanosphere, a silica nanoparticle, and/or a microbubble.

As used herein, the terms “vehicle binding” or “bound vehicle” or “binding” are used to refer to a single time or reproducible interaction between the vehicle and a host cell, more specifically, the surface or outer membrane of the host cell. Vehicle binding may comprise, but is not limited to, ligand-targeted binding, binding facilitated by a chemical interaction, charge, receptor-mediated binding, hydrophobicity, or viral binding to a host cell.

### Device

Provided herein are devices, methods, and compositions for the treatment of a disease or disorder comprising a closed-loop, continuous flow system. The devices and methods may allow for patient-connected bedside delivery of a payload to a subject extracorporeally in a single, convenient outpatient procedure, avoiding the complexity of traditional methods of delivering cellular therapies. The devices described herein comprise multiple modules, including at least an apheresis module and a binding module, each of which is described in more detail below. The system may include cell processing to wash, buffer exchange and removal of free vehicle or payload before reintroduction to patient.

Generally, in an aspect, the device described herein comprises an apheresis module capable of collecting peripheral blood mononuclear cells (PBMCs) in a process termed leukapheresis. The apheresis module may comprise an apheresis module known in the art or may be custom designed. The device described herein also comprises a binding module. In some embodiments, the apheresis module and the binding module operate as a single unit. In some embodiments, the apheresis module and the binding module are connected to form a single closed-loop fluid pathway. In some embodiments, the disposable set fluid pathway is connected to the subject's circulatory system via venepuncture for the duration of the extracorporeal system procedure.

### Apheresis Module

In an embodiment, the subject is directly connected to the device, specifically the apheresis module. In an embodiment, the apheresis module comprises a leukapheresis system to separate PBMCs (e.g., the Fresenius Kabi Amicus Separator™ or a Terumo Optia™) or subtypes of PBMCs (e.g., T cells, B cells, monocytes, and NK cells) from the plasma and red blood cells. The number of PBMCs or cells of a certain subtype of PBMC can be standardized by altering the processed blood volume based on the patient's complete blood count (CBC) at time of collection.

### Binding Module

The devices described herein also comprise a binding module, in which the subject-derived cells are contacted with a payload to form a population of PACCs. The binding module may facilitate binding of the cells to a payload. In an embodiment, the payload is packaged with or in a delivery vehicle, such as a lipid nanoparticle. In an embodiment, the delivery vehicle is a polymeric nanoparticle. In an embodiment, the delivery vehicle is a viral vector. In an embodiment, the delivery vehicle is a vesicle including, but not limited to, an extracellular vesicle (EV), a microvesicle (MV), an exosome, or exosome-like vesicle (ELV). In an embodiment, the delivery vehicle is introduced to the cell or binding module via a sterile port. In an embodiment, the delivery vehicle is introduced via a sterile port using a syringe.

In an embodiment, the plasma is removed from the sample. In an embodiment, the plasma is exchanged with a binding buffer. In an embodiment, the binding buffer is a buffer that increases the binding of a payload delivery vehicle. In an embodiment, the binding buffer is a buffer that decreases the binding of a payload delivery vehicle to increase selective binding. The binding buffer utilized in the method as disclosed herein can be a binding buffer known in the art. In some embodiments, the binding buffer is optimized (e.g., pH, ionic strength, comprising proteins and/or peptides, tonicity) for efficient binding of a payload delivery vehicle to a target cell (e.g., a PBMC).

In certain embodiments, the concentration of cells in an apheresis sample is determined. In an embodiment, the cells are diluted in order to obtain a lower concentration of cells. In an embodiment, the cells are concentrated in order to obtain a higher concentration of cells. In an embodiment, the desired concentration of cells will be dependent on payload being delivered and the disease being treated. In an embodiment, the cells are concentrated within the apheresis module. In an embodiment, the separated cells are shunted to a binding module.

In an embodiment, the binding system comprises a binding chamber. The binding chamber contains the cell sample collected and separated during apheresis or sample collected and separated during apheresis that has been further prepared for binding. In an embodiment, the binding chamber is a medical grade blood collection bag. In an embodiment, the binding chamber is a cell culture chamber or flask. In an embodiment, the binding chamber is a beaker. In an embodiment, the binding chamber is set on a binding chamber platform. The binding chamber platform allows the cells to mix with the payload delivery vehicle. Mixing can be performed by shaking, tilting, rotating, spinning, or other mode to optimize vehicle contact and binding with cells.

In an embodiment, the cells and the payload delivery vehicle are mixed at concentration ratios optimized for efficient binding of the payload delivery vehicle to the cells. In an embodiment, a higher ratio of cells to payload delivery vehicle is used. In an embodiment, a higher ratio of payload delivery vehicle to cells is used. In an embodiment, the concentration of cells to payload delivery vehicle are equal (e.g., a 1:1 ratio).

In an embodiment, the binding chamber platform controls the temperature of incubation of the cells and the payload delivery vehicle to optimize binding of the payload delivery vehicle and the cells, thereby forming stable PACCs. In an embodiment, incubation occurs between about 0° C. to about 40° C. In an embodiment, incubation occurs between about 0° C. to about 10° C. In an embodiment, incubation occurs between about 0° C. to about 2° C. In an embodiment, incubation occurs between about 2° C. to about 4° C. In an embodiment, incubation occurs between about 4° C. to about 6° C. In an embodiment, incubation occurs between about 3° C. to about 7° C. In an embodiment, incubation occurs between about 6° C. to about 8° C. In an embodiment, incubation occurs between about 2° C. to about 8° C. In an embodiment, incubation occurs between about 8° C. to about 10° C. In an embodiment, incubation occurs between about 10° C. to about 15° C. In an embodiment, incubation occurs between about 15° C. to about 20° C. In an embodiment, incubation occurs between about 20° C. to about 25° C. In an embodiment, incubation occurs between about 25° C. to about 30° C. In an embodiment, incubation occurs between about 30° C. to about 37° C. In an embodiment, incubation occurs at about 4° C., about 5° ,. about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36, about 37° C. In an embodiment, the incubation occurs at 0° C. In an embodiment, the incubation occurs at 4° C. In an embodiment, the incubation occurs at 10° C. In an embodiment, the incubation occurs at 15° C. In an embodiment, the incubation occurs at 22° C. In an embodiment, the incubation occurs at 25° C. In an embodiment, the incubation occurs at 37° C. In an embodiment, the incubation occurs at 40° C.

In an embodiment, incubation of the cells and the payload delivery vehicle within the binding chamber occurs for a sufficient amount of time that allows for the binding of the payload delivery vehicle and the cells, thereby forming stable PACCs. In an embodiment, incubation time is between about 1 minute and about 180 minutes. In an embodiment, incubation time is between about 1 minutes and about 20 minutes. In an embodiment, incubation time is between about 1 minutes and about 10 minutes. In an embodiment, incubation time is between about 5 minutes and about 30 minutes. In an embodiment, incubation time is between about 30 minutes and about 45 minutes. In an embodiment, incubation time is between about 45 minutes and about 60 minutes. In an embodiment, incubation time is between about 60 minutes and about 75 minutes. In an embodiment, incubation time is between about 75 minutes and about 90 minutes. In an embodiment, incubation time is between about 90 minutes and about 105 minutes. In an embodiment, incubation time is between about 105 minutes and about 120 minutes. In an embodiment, incubation time is between about 120 minutes and about 135 minutes. In an embodiment, incubation time is between about 135 minutes and about 150 minutes. In an embodiment, incubation time is between about 150 minutes and about 165 minutes. In an embodiment, incubation time is between about 165 minutes and about 180 minutes.

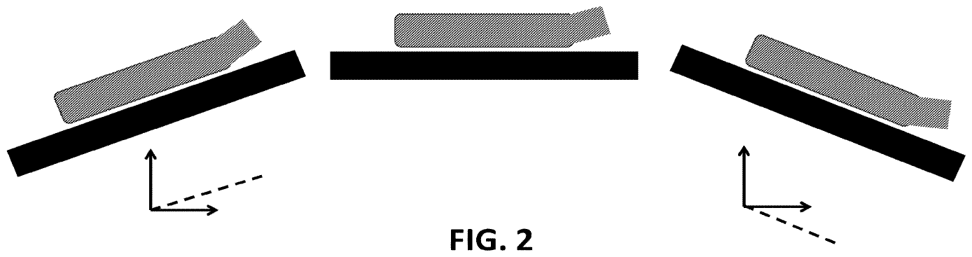

In an embodiment, the binding chamber platform mixes the cell and payload delivery vehicle mixture. In an embodiment, the mixing allows for binding of the payload delivery vehicle to the cell while preserving cell viability (e.g., not damaging the cells during mixing). In an embodiment, the platform is a rocker of a specific angle. In an embodiment, the angle is between 20° and 80°, in both directions. In an embodiment, the angle is between about 20° and about 25°, in both directions. In an embodiment, the angle is between about 25° and about 30°, in both directions. In an embodiment, the angle is between about 30° and about 35°, in both directions. In an embodiment, the angle is between about 35° and about 40°, in both directions. In an embodiment the angle is between about 40° and about 45°, in both directions. In an embodiment, the angle is between about 45° and about 50°, in both directions. In an embodiment, the angle is between about 50° and about 55°, in both directions. In an embodiment, the angle is between about 55° and about 60°, in both directions. In an embodiment, the angle is between about 60° and about 65°, in both directions. In an embodiment, the angle is between about 65° and about 70°, in both directions. In an embodiment, the angle is between about 70° and about 75°, in both directions. In an embodiment, the angle is between about 75° and 80°, in both directions. In an embodiment, the angle is 30°, in both directions. In other embodiments, the positive and negative angle may be of different degrees of any combination between 20° and 80°, in both directions. An exemplary agitation scheme is disclosed, for example, in FIG. 2.

In an embodiment, the binding chamber platform is a nutator mixer. In an embodiment, the binding chamber platform is an orbital mixer. In an embodiment, the binding chamber platform in a shaker mixer. In an embodiment, the rate of mixing and duration may be variable. In an embodiment, the binding chamber platform is an agitator. In an embodiment, the binding chamber platform facilitates mixing of the cells and the payload delivery vehicle during incubation in the binding chamber. In an embodiment, mixing of the cells and the payload delivery vehicle during incubation is constant. In an embodiment, mixing of the cells and the payload delivery vehicle during incubation is intermittent.

In an embodiment, the apheresis module is directly linked to the binding module. In an embodiment, the apheresis module and binding module may be separated by one or more processing modules responsible for processing the sample, such as washing the sample, storing the sample. In an embodiment, the devices described herein comprises at least 1, 2, 3, 4, 5, 6 or more processing modules (e.g., washing modules).

### Processing Modules

In an embodiment, the cells are washed in a washing module after incubation in the binding chamber. During this step, the cells may be washed and free payload delivery vehicles (e.g., vehicles that are not bound to a cell) are removed from the cell sample. In an embodiment, the washing step comprises centrifugation of the cells. In an embodiment, the washing step comprises filtration of the cells. In an embodiment, the washing step comprises spinning membrane filtration. In an embodiment, the washing step comprises density gradient centrifugation. In an embodiment, the washing step comprises immuno-density cell isolation.

In an embodiment, the binding buffer is exchanged with reinfusion buffer. In an embodiment, the reinfusion buffer is a saline buffer. In an embodiment, the saline buffer comprises between about 1 g of sodium chloride per liter of water (0.1% saline) to about 10 g of sodium chloride per liter of water (1.0% saline). In an embodiment, the saline buffer is between about 0.1% to about 0.2% saline. In an embodiment, the saline buffer comprises between about 0.2% to about 0.3% saline. In an embodiment, the saline buffer comprises between about 0.3% to about 0.4% saline. In an embodiment, the saline buffer comprises between about 0.4% to about 0.5% saline. In an embodiment, the saline buffer comprises between about 0.5% saline and about 0.6% saline. In an embodiment, the saline buffer comprises between about 0.6% to about 0.7% saline. In an embodiment, the saline buffer comprises between about 0.7% to about 0.8% saline. In an embodiment, the saline buffer comprises between about 0.8% to about 0.9% saline. In an embodiment, the saline buffer comprises between about 0.9% to about 1.0% saline. In an embodiment, the saline buffer comprises about 0.9% saline.

In some embodiments, the reinfusion buffer may comprise any buffer commonly used for intravenous administration known in the art. Such buffers may include 0.9% saline, PlasmaLyte, Ringer's solution, and dextrose-buffered saline; such buffers may be formulated with or without addition of additives such as human serum albumin (HSA).

In an embodiment, after the binding buffer is exchanged with reinfusion buffer and the cells are ready for reinfusion into the patient, the cells are reinfused into the patient. In an embodiment, the cells are reinfused into the patient through the same initial port. In an embodiment, the cells are reinfused into the patient through a different port. In an embodiment, the patient is connected to the system throughout the entirety of the process.

In an embodiment, the methods described herein features a biphasic temperature, wherein the first phase comprises a temperature sufficient for forming PACCS, e.g., as described above, and the second phase comprises a series of increases in temperature between the first phase and the subject's body temperature. For example, in some embodiments, the first phase comprises a temperature of between about 0° C. to about 40° C. In some embodiments, the first phrase comprises a temperature of between about 0° C. to about 37° C. In some embodiments, the first phrase comprises a temperature of between about 0° C. to about 25° C. In some embodiments, the first phrase comprises a temperature of between about 0° C. to about 22° C. In some embodiments, the first phrase comprises a temperature of between about 0° C. to about 10° C. In some embodiments, the first phrase comprises a temperature of between about 0° C. to about 4° C. In some embodiments, the first phrase comprises a temperature of between about 0° C. to about 10° C. In some embodiments, the first phrase comprises a temperature of between about 10° C. to about 22° C. In some embodiments, the first phrase comprises a temperature of between about 10° C. to about 25° C. In some embodiments, the first phrase comprises a temperature of between about 10° C. to about 37° C. In some embodiments, the first phrase comprises a temperature of about 0° C. In some embodiments, the first phrase comprises a temperature of about 4° C. In some embodiments, the first phrase comprises a temperature of about 10° C. In some embodiments, the first phrase comprises a temperature of about 22° C. In some embodiments, the first phrase comprises a temperature of about 25° C. In some embodiments, the first phrase comprises a temperature of about 37° C. In some embodiments, the increases in temperature in the second phase correspond with increments of 5° C. or 10° C.

### PACC Testing and Verification

In an embodiment, samples before and after binding are obtained. In an embodiment, a sample is taken from the patient. In an embodiment, a sample is taken from the patient before apheresis. In an embodiment, the sample is assayed for cell type and cell health (e.g., cellularity (CBC), cell viability, cell count). In an embodiment, the sample is assayed for cell markers indicated various immune cell subtypes (e.g., B cells, monocytes, hematopoietic stem cells (HSCs), T cells, NK cells). In an embodiment, a sample is taken after apheresis (e.g., leukapheresis). In an embodiment, a sample is obtained to determine cell counts. In an embodiment, cell counts are collected to determine cell concentration. In an embodiment, a sample is obtained to determine cellularity. In an embodiment, a sample is obtained to determine cell viability. In an embodiment, a sample is taken after the plasma is exchanged with the binding buffer. In an embodiment, samples are taken after introduction of a payload delivery vehicle to the cells. In an embodiment, the samples taken after introduction of a payload delivery vehicle at various time points. In an embodiment, a sample is taken at the completion of the binding step. In an embodiment, the sample taken at the completion of the binding step may be assayed for cell count, cell viability, cell type (e.g., detection of cell markers), and binding efficiency. In an embodiment, a sample of PACCs may be placed in an incubator to mimic in vivo conditions to assess uptake of payload, transcription and/or translation of the payload (e.g., mRNA), cell viability or other parameters useful in assessing or predicting clinical outcomes. In an embodiment, a sample is taken after the cells are washed and free payload delivery vehicles (e.g., vehicles that are not bound to a cell) are removed from the cell sample. In an embodiment, a sample may be taken after the sample is washed and resuspended in an infusion buffer prior to return to the patient. In an embodiment a sample may be taken at various timepoints during the return to the patient. In an embodiment a post-procedure patient sample may be taken after the patient has been disconnected from the system.

In some embodiments, cell enrichment prior to payload introduction is contemplated. In certain embodiments, cells may be enriched based on physiological properties such as density, size, acoustics, charge, binding properties, etc. Methods and systems employing surface protein-based target cell separation/purification techniques are often included and/or preferred. In such methods, a targeting moiety is utilized to specifically bind a cell surface protein on a known or predetermined cell population (including mixtures of cell populations sharing that cell surface protein). Often in such techniques a targeting moiety such as an antibody or binding fragment thereof is bound on or to a support (e.g., microbubble, magnetic particle, nanoparticle, bead, etc.) and the targeting moiety-bound support is introduced to a sample and permitted to mix within the sample and become bound with one or more target cells in the sample. Thereafter a process is used to separate or otherwise remove the support-bound cell from the remainder of the sample.

The present methods and systems contemplate an overarching requirement to not alter or harm the structure or function of the target cells nor adversely affect the medium in which they exist though that medium may be altered or replaced. Moreover, the present methods and systems are directed to increasing the specificity and speed with which target cells are separated/purified.

In any and all of the aspects as described herein, a patient is coupled to the system in a closed-loop fashion, with an inlet conduit coupled or connected to the patient to provide peripheral blood as input to the system. The inlet conduit receives blood directly from the circulation of the patient which is passed to the cell purification system. The cell separation module is designed, at a minimum, to perform collection of specific or known cell populations or collections present in the blood. This includes cell separation from blood components and optionally one or more enrichment steps. The cell purification systems contemplated herein are often adapted to purify cells from blood or other body fluids based on surface protein expression, size, shape, granularity, buoyancy, density, genetic identification, or any combination thereof. Mechanisms for cell separation/purification often according to the presently included methods and systems include buoyancy, centrifugal force, filtration, elutriation, sonic, electrical, magnetic, and/or acoustic properties.

The systems and approaches described herein can also involve one or more steps of cell selection to isolate or enrich for cells that have been modified in a connected system.

In some embodiments, the system can execute a program of cell purification in an automated manner with no or minimal operator input once the program is initiated.

In conjunction with any or all of the techniques described in further detail below, filtration is or can be used as a method of reducing the volume of samples and/or separating sample components based on their ability to flow through or be retained by the filter. Sample centrifugation and/or leukapheresis (or aspects thereof) may be part of this sample treatment. Filtration, sample centrifugation and/or leukapheresis (or aspects thereof) may occur before and/or after target cell separation/purification.

Timing is considered an important overall aspect of the system as disclosed herein. As the system is parenterally patient-connected, it is important that the time the system is parenterally connected with the patient be kept within tolerated periods of time. All contemplated system embodiments herein are configured with this aspect in mind. Tolerance to prolonged connection with the system is understood to be variable, with an understanding of the present inventors that reducing this total time period and maximizing the effectiveness of the present systems within that time period results in inter-module cycle time period reductions and better tolerance of the patients/subjects to the treatment protocol delivered by the systems. As such the present systems are configured to operate within target cycle times. This configuration relates to the purification technologies and modules utilized, the detector mechanisms and functionalities, the disposable aspects of the system, reagents, software, and/or the structural and/or functional arrangement of the system to ensure efficient and safe operation and quick passage of blood/sample/target cells within the systems and between or within modules. Overall, a treatment protocol, beginning with the presently described closed-loop parenteral connection of the patient with the system and ending with removal of the parenteral connection from the patent, will last between 1 to 6 hours. Most frequently this time period is between 2 to 4 hours. Within these ranges there are subranges as contemplated in the embodiments of the present systems and their operation. These ranges may be anywhere in the range of at or about 1 hour, at or about 1.5 hours, at or about 2 hours, at or about 2.5 hours, at or about 3 hours, at or about 3.5 hours, at or about 4 hours, at or about 4.5 hours, at or about 5 hours, at or about 5.5 hours, at or about 6 hours, or shorter than 2 hours. In certain limited embodiments the time period may be above 6 hours. It is noted that there are a variety of patient safety issues that are balanced in the process of the automated manner the present systems are configured and how they operate. In this regard, timing and safety considerations are balanced to ensure not only that the proper or desired treatment is provided in a quick and efficient manner, but also that safety protocols are rigorously adhered to in the system to ensure that no harm is caused by the operation of the system. Such issues related to the safety of modules, sample processing procedures, and reagents are discussed herein and integrated in the most frequent embodiments.

While a variety of cell purification methods and systems are contemplated herein, it is appreciated by the inventors that not all available cell purifications are suitable for the patient-connected systems of the present disclosure. For example, fluorescence-activated cell sorting, laser capture microdissection, methods dependent on cell culture and differentiation, methods dependent on incubation of cells with other cells or materials that are or could be harmful to the patient, methods dependent on attachment to biomaterials such as fibronectin, among other related and similar methods having similar or related drawbacks are not contemplated as part of the systems and apparatus disclosed herein.

Further, the presently contemplated systems and methods employ cell purification methods and systems that permit immediate passage of “processed cells” (i.e., target cell populations that have been purified from non-target cells or other blood components) to a cell customization module or the next step or module in the system. In this regard and simply as one example, conventional DYNAL® (Dynal, A.S., Oslo, Norway) bead-based methods are not suitable in the present methods and systems. For example, such conventional DYNAL bead-based methods and reagents require incubation with target cells over a prolonged period such as multiple days. Eventually, after this prolonged time period or an additional time period, the Dynal beads detach from the target cells. Transduction of the target cells is not possible until after the beads detach from the target cells, thus prolonging the process and rendering a patient-connected system employing such methods and reagents unsuitable and not previously possible. While DYNAL beads are specifically discussed, the concept of research lab tools, which tools require long time periods (e.g., hours to days) to process, being unsuitable for the presently described systems and methods is intended to be a general statement. This holds true with regard to these or other conventional beads that require a prolonged time period of incubation, or non-biocompatible or harmful reagents, for connection with a target cell. This also holds true with regard to these or other conventional beads that require a prolonged time period of incubation/waiting, non-biocompatible or harmful reagents, or cell viability or function disruptive methods or reagents, for detachment from a target cell. Each of these methods and reagents is considered to fall outside the presently contemplated embodiments as not compatible with the contemplated patient-connected (including closed-loop) systems.

Blood from a patient into the presently contemplated systems is typically received in an apheresis unit or module or leukapheresis module or unit prior to purification. A variety of apheresis or leukapheresis technologies and devices may be employed. For example, non-limiting examples of currently used apheresis devices include, for example: COBER Spectra, TRIMA®, and SPECTRA OPTIA® systems (all marketed by Terumo) and the AMICUS® and COM.TEC® (marketed by Fresenius Kabi) devices.

In various embodiments, a cell washing unit can be integrated with the presently described systems, for example, placed between an apheresis module and the cell purification module and/or after the cell purification module, to remove one or more components of a cell suspension produced by the apheresis module or cell purification module, or in order to place the cells into a medium or solution better conducive to cell enrichment procedure. A cell washing unit generally includes a source of cell wash solution, such as a reservoir or bag of wash solution, connected via conduit or tubing to the washing unit. The flow of cell wash solution can be controlled by a valve controlled by a processor. A cell washing unit can include a centrifugal unit similar to the centrifugal unit in an apheresis device; cells enter the washing unit, are mixed with wash solution introduced from a reservoir or other source of wash solution via conduit connected to the washing unit, and the suspension is spun to concentrate cells. One or more rounds of cell washing and re-suspension can be performed before the cells pass out of the cell washing unit. A cell washing unit can be interfaced with a detector configured to detect one or more properties of the cells or cell suspension, e.g., cell number, solution density, solution pH, solution ionic strength, etc.

Other stand-alone devices, such as the Gambro COBE 2991™ Blood Cell Processor, the Fresenius Kabi Lovo®, the Fresenius Kabi Cue®, the Baxter CYTOMATE™, or elutriation devices such as Rotea™ marketed by ThermoFisher. Cell Washing System are often used to wash, concentrate, or place cells into appropriate concentration and medium prior to purification. Devices of this kind can be adapted for or integrated into a system as described herein for bedside customization of nucleated blood cells.

The cell purification system often advantageously utilizes buoyancy such as microbubble separation of target cells or one or more cell population from other blood or sample components. For example, highly efficient component separation may be achieved using flotation methods involving binding target cells with encapsulated gas microbubbles. The efficiency of such separations is enhanced by the substantial density difference between gas microbubbles and liquid sample media, so that the process is capable of high sensitivity. Flotation separations inherently proceed more gently than magnetic separations and less encumbered by residual materials more critical in patient connected systems. Gas microbubbles advantageously are prepared using flexible encapsulating materials, so that the possibility of causing damage to sensitive target components such as cells during separation may thus be minimized. Unlike some magnetic systems such as DYNAL, microbubbles can be made from biomaterials that are safe for re-administration back to patients which is critical for patient-connected systems. DYNAL and other bead systems used in centralized manufacturing have the time and mechanisms to ensure 100% removal of the beads prior to final formulation that is delivered back to the patient which is not possible in a patient-connected manner. Microbubbles also permits easy removal of the microbubbles from the target cell after its use is complete, for example by bursting the microbubbles. Therefore, a process for the separation of target material from a liquid sample is provided that includes coupling a target cell to a microbubble, allowing the bound microbubble to rise in the surrounding fluid optionally to a specific area, and then removing the microbubble from the target cell (positive selection) or recovering target-free sample material (negative selection) of the cells that were not bound to the targeting microbubbles. The process of incubating cells with microbubbles and mixing/agitating the combined cells with microbubbles to provide for attachment of the microbubbles with cells is generally defined by a predetermined time duration, for example, between at or about 5 minutes to at or about 15 minutes. This timeframe is critical as much longer are not possible in patient-connected systems. In certain often included embodiments, the incubation and/or agitation time is less than at or about 15 minutes. In certain embodiments, the incubation and/or agitation time is greater than at or about 15 minutes. In certain embodiments, the incubation and/or agitation time is less than at or about 10 minutes. In certain embodiments, the incubation and/or agitation time is less than at or about 5 minutes. In certain embodiments, the incubation and/or agitation time is between at or about 5 minutes to at or about 10 minutes.

The process of incubating cells with microbubbles and mixing/agitating the combined cells with microbubbles to provide for attachment of the microbubbles with cells is generally defined by a predetermined temperature, for example, between at or about 0° C. to at or about 40° C. In certain often included embodiments, the incubation and/or agitation temperature is between at or about 10° C. to at or about 30° C. In certain embodiments, the incubation and/or agitation temperature is between at or about 15° C. to at or about 25° C. These temperature ranges are often preferred in patient-connected systems.

Microbubbles that are useful in accordance with the present systems and methods include any microbubbles that can be prepared in a targetable form. In general, in the present systems and methods, the encapsulating material and gas content are both biocompatible.

Microbubble encapsulating materials (or shell material) suitable for buoyancy-based separation is generally composed of biocompatible and bioinert materials. This includes being inert relative to the target cells, and any contamination that results does not affect downstream system processes or have an in vivo effect when passed back into a patient in the closed loop. In this regard, the anionic lipid phosphatidylserine is often avoided. Moreover, other lipids commonly taught in the context of microbubble shell (for example, phosphatidic acids, phosphatidylinositol, cardiolipins, sphingomyelins), which participate in cell signaling, are on the less preferred list of encapsulation materials.

Representative examples of encapsulating materials include a coalescence-resistant surface membrane (e.g., gelatin), a filmogenic protein (e.g., albumin, gamma globulin, apotransferrin, hemoglobin, collagen, urease, human serum albumin, etc.), a filmogenic protein mixed with a polymer (e.g., albumin/dextran), a polymer material (natural or synthetic or modified), an elastic interfacial synthetic polymer membrane, a microparticulate biodegradable polyaldehyde, a microparticulate N-dicarboxylic acid derivative of a polyamino acid polycyclic imide, a lipid, a protein, or a surfactant. In some embodiments, the encapsulating material may be a coalescence-resistant surface membrane (e.g., gelatin). In some embodiments, the encapsulating material may be a filmogenic protein (e.g., albumin, gamma globulin, apotransferrin, hemoglobin, collagen, urease, human serum albumin, etc.). In some embodiments, the encapsulating material may be a filmogenic protein mixed with a polymer (e.g., albumin/dextran). In some embodiments, the encapsulating material may be a polymer material (natural or synthetic or modified). In some embodiments, the encapsulating material may be an elastic interfacial synthetic polymer membrane. In some embodiments, the encapsulating material may be a microparticulate biodegradable polyaldehyde. In some embodiments, the encapsulating material may be a microparticulate N-dicarboxylic acid derivative of a polyamino acid-polycyclic imide, a lipid, a protein, or a surfactant. The encapsulating material may be a phospholipid and/or a lipopeptide.

Exemplary shell forming lipids that present no net charge and may be utilized include phosphatidylcholines, in particular disteroylphosphatidylcholine, dipalmitoylphosphatidylcholine, and dimyrstylphosphatidylcholine), disteroylphosphatidylethanolamines, fatty acids, in particular stearic acid and palmitic acid, PEGylated ceramides, and PEGylated fatty acids. Often the microbubbles contain polypeptides, including a specific peptide, polypeptide, protein or combinations thereof (including multiple different types of each category). Synthetic and naturally occurring peptides, polypeptides, proteins or combinations thereof are contemplated.

In some embodiments, the microbubble comprises a targeting moiety. In some embodiments, the microbubble comprises more than one targeting moiety. In some embodiments, the targeting moiety is a protein. In some embodiments, the protein is an antibody or an antibody fragment thereof, such as an antigen-binding fragment thereof. In some embodiments, the antibody or antigen-binding fragment thereof may comprise CD3, CD4, CD5, CD6, CD8, CD14, CD16, CD19, CD20, CD34, CD56, and/or CD117. For example, in some embodiments, the antibody or antigen-binding fragment thereof may comprise CD3. In some embodiments, the antibody or antigen-binding fragment thereof may comprise CD4. In some embodiments, the antibody or antigen-binding fragment thereof may comprise CD5. In some embodiments, the antibody or antigen-binding fragment thereof may comprise CD6. In some embodiments, the antibody or antigen-binding fragment thereof may comprise CD8. In some embodiments, the antibody or antigen-binding fragment thereof may comprise CD14. In some embodiments, the antibody or antigen-binding fragment thereof may comprise CD16. In some embodiments, the antibody or antigen-binding fragment thereof may comprise CD19. In some embodiments, the antibody or antigen-binding fragment thereof may comprise CD20. In some embodiments, the antibody or antigen-binding fragment thereof may comprise CD34. In some embodiments, the antibody or antigen-binding fragment thereof may comprise CD56. In some embodiments, the antibody or antigen-binding fragment thereof may comprise CD117. In some embodiments, the microbubble can be activated in vivo. In some embodiments, the microbubble is activated using focused ultrasound.

### Target Cells

In one aspect, a payload delivery vehicle targets a cell in a subject. In some embodiments, the subject is a mammal. In some embodiments, the mammal is a human. In some embodiments, the cell is isolated from a type of tissue. In some embodiments, the tissue is from a particular organ. In some embodiments, the organ or tissue is any of the following: brain, spinal cord, liver, blood, epidermis, neural, bone, kidney, cardiac, lung, endocrine, connective, muscle, or endothelial. In some embodiments, the organ or tissue is derived from peripheral blood.

### Immune Cells

In certain embodiments, the target cell is an immune cell. In some embodiments, the immune cell is a T cell, e.g., a CD8+ T cell, a CD4+ T cell, a regulatory T cell (Treg), a stem cell memory T cell, a lymphoid progenitor cell, a hematopoietic stem cell (HSC), a natural killer cell (NK cell), a natural killer T cell (NKT cell) or B cell. In some embodiments, the cells are myeloid cells e.g., monocytes, macrophages, dendritic cells, or granulocytes e.g., neutrophils, mast cells, eosinophils, and/or basophils.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive Treg cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, and/or engineering them. In some embodiments, preparation of the engineered cells includes one or more separation/preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, washing, and/or incubation and contacting of the payload vehicle to the cell. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

### Payloads

### Nucleic Acids

In some embodiments, the payload is a nucleic acid including, for example, messenger RNA (mRNA), antisense oligonucleotides, plasmid DNA, microRNA (miRNA), miRNA inhibitors (antagomir/antamir), messenger-RNA-interference complementary RNA (iRNA), DNA, multivalent RNA, dicer substrate RNA, complementary DNA (cDNA), transfer RNA (tRNA), ribosomal RNA (rRNA), modified nucleic acids, and the like. In some embodiments, the nucleic acid is comprised in a nanoparticle to deliver said nucleic acid to a target cell to generate target cells that produce an exogenous polypeptide of interest, or to alter expression of an endogenous polypeptide of interest. In some embodiments, the nanoparticle can be any as described herein.

In some embodiments, the nucleic acid is modified. In some embodiments, the modification comprises a non-canonical nucleotide. Certain non-canonical nucleotides, when incorporated into synthetic RNA molecules, can reduce the toxicity of the synthetic RNA molecules, in part by interfering with binding of proteins that detect exogenous nucleic acids. Non-canonical nucleotides that have been reported to reduce the toxicity of synthetic RNA molecules when incorporated therein include: pseudouridine, 5-methyluridine, 2-thiouridine, 5-methylcytidine, N6-methyladenosine, and certain combinations thereof. However, the chemical characteristics of non-canonical nucleotides that can enable them to lower the in vivo toxicity of synthetic RNA molecules have, until this point, remained unknown. Furthermore, incorporation of large amounts of most non-canonical nucleotides, for example, 5-methyluridine, 2-thiouridine, 5-methylcytidine, and N6-methyladenosine, can reduce the efficiency with which synthetic RNA molecules can be translated into protein, limiting the utility of synthetic RNA molecules containing these nucleotides in applications that require protein expression. In addition, while pseudouridine can be completely substituted for uridine in synthetic RNA molecules without reducing the efficiency with which the synthetic RNA molecules can be translated into protein, in certain situations, for example, when performing frequent, repeated transfections, synthetic RNA molecules containing only adenosine, guanosine, cytidine, and pseudouridine can exhibit excessive toxicity.

In some embodiments, the payload is an antisense oligonucleotide (ASO), or antisense compounds. In general, antisense compounds hybridize to a target nucleic acid and effects modulation of gene expression activity or function, such as transcription, translation or splicing. The modulation of gene expression can be achieved by, for example, target degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi is a form of antisense-mediated gene silencing involving the introduction of dsRNA leading to the sequence-specific reduction of targeted endogenous mRNA levels. This sequence-specificity makes antisense compounds extremely attractive as tools for target validation and gene functionalization, as well as therapeutics to selectively modulate the expression of genes involved in the pathogenesis of any one of a variety of diseases.

Antisense technology is an effective means for reducing the expression of one or more specific gene products and can therefore prove to be uniquely useful in a number of therapeutic, diagnostic, and research applications. Chemically modified nucleosides are routinely used for incorporation into antisense compounds to enhance one or more properties, such as nuclease resistance, pharmacokinetics or affinity for a target RNA.

In some embodiments, the nucleic acid is a circular or circularized nucleic acid. In some embodiments, the circular or circularized nucleic acid comprises DNA. In some embodiments, the circular or circularized nucleic acid comprises RNA. Circular RNA is useful in the design and production of stable forms of RNA. The circularization of an RNA molecule provides an advantage to the study of RNA structure and function, especially in the case of molecules that are prone to folding in an inactive conformation (Wang and Ruffner, 1998). Circular RNA can also be particularly interesting and useful for in vivo applications, especially in the research area of RNA-based control of gene expression and therapeutics, including protein replacement therapy and vaccination. In some embodiments, the circular or circularized RNA can be, but not limited to, any as described in PCT/US2020/063494, PCT/US2020/034418, PCT/US2021/031629, or PCT/US2021/033276, which are incorporated herein by reference in their entireties.

In some embodiments, the nucleic acids as described herein are modified nucleic acids. In some embodiments, the modified nucleic acids comprise modified polynucleotide backbones. Modified polynucleotide backbones have been shown to improve half-life as well as membrane penetration. Variations on polynucleotide backbones include the use of methylphosphonates, monothiophosphates, dithiophosphates, phosphoramidates, phosphate esters, bridged phosphoroamidates, bridged phosphorothioates, bridged methylenephosphonates, dephospho internucleotide analogs with siloxane bridges, carbonate bridges, carboxymethyl ester bridges, acetamide bridges, carbamate bridges, thioether, sulfoxy, sulfono bridges, various “plastic” DNAs, α-anomeric bridges, and borane derivatives. In some embodiments, the modified polynucleotide backbone is a polyamide backbone. In some embodiments, the modified polynucleotide backbone comprises an extra bridge connecting the 2′ oxygen and 4′ carbon (e.g., a locked nucleic acid (LNA)). In some embodiments, the modified nucleic acids comprise modifications on the base. In some embodiments, the modification is a methyl group. In some embodiments, the modification is a sugar. In some embodiments, the modified nucleic acids comprise modifications to the phosphate backbone of the nucleic acid.

### Antibodies and Antigen-Binding Fragments

In some embodiments, the payload is, or encodes, an antibody. An antibody is an immunoglobulin or immunoglobulin-like molecule, including, but not limited to, IgA, IgD, IgE, IgG, and IgM, combinations thereof, and includes similar molecules produced during the immune response in any vertebrate (e.g., humans, goats, rabbits, and mice, and non-mammalian species such as shark immunoglobulins). The antibody can be an intact immunoglobulin and/or an antibody fragment or antigen-binding fragment that specifically binds to a molecule of interest (or a group of highly similar molecules of interest) to the substantial exclusion of binding to other molecules (for example, antibodies and antibody fragments that have a binding constant for the molecule of interest that is at least 10³ M−1 greater, at least 10⁴ M−1 greater or at least 10⁵ M−1 greater than a binding constant for other molecules in a biological sample). In some embodiments, the antibody is an engineered antibody, for example, a chimeric antibody (e.g., humanized murine antibodies). In some embodiments, the antibody is a heteroconjugate antibody (e.g., a bispecific antibody). See also, Pierce Catalog and Handbook, 1994-1995 (Pierce Chemical Co., Rockford, Ill.); Kuby, J., Immunology 3rd unology, Ed., W. H. Freeman & Co., New York, 1997.

Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds a particular antigen will have a specific VH region and the VL region sequence, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen-binding. These positions within the CDRs are called specificity determining residues (SDRs).

In some embodiments, the payload is, or encodes, an antigen-binding fragment. In some embodiments, the antigen-binding fragment is a digestion fragment, specified portions, derivatives, and variants thereof, including antibody mimetics or comprising portions of antibodies that mimic the structure and/or function of an antibody or specified fragment or portion thereof, including single chain antibodies and fragments thereof. In some embodiments, the antigen-binding fragment is selected from the group of a single chain variable fragment (scFv), fragment antigen-binding region (Fab), F(ab′)2, Fab′, a monovalent fragment consisting of the VL, VH, CL, and CH domains, or a variable fragment (Fv). Although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl.

Acad Sci. USA 85:5879-5883. In some embodiments, the antigen-binding fragment is a single domain antibody (dAb) fragment (Ward et al. (1989) Nature 341:544-546), which consists of a VH domain; and an isolated complementarity determining region (CDR). Any of the above-noted antibody fragments can be obtained using conventional techniques known to those of skill in the art, and the fragments are screened for binding specificity and neutralization activity in the same manner as are intact antibodies.

### Immune Receptors

In some embodiments, the payload is, or encodes, an immune receptor. Traditional cancer treatments mainly comprise surgery, radiotherapy, chemotherapy, and stem cell transplants.

In some embodiments, the immune receptor comprises a target-specific binding element, e.g., an antigen-binding domain. The choice of antigen-binding element depends upon the type and number of ligands that define the surface of a target cell. For example, the antigen-binding domain may be chosen to recognize a ligand that acts as a cell surface marker on target cells associated with a particular disease state. Thus, examples of cell surface markers that may act as ligands for the antigen moiety domain in the CAR of the invention include those associated with viral, bacterial and parasitic infections, autoimmune disease, alloimmune disease, and cancer cells.

In some embodiments, the immune receptor can be engineered to target an antigen of interest by way of engineering a desired antigen-binding domain that specifically binds to an antigen on a tumor cell or a cell associated with a hyperproliferative disorder, e.g., cancer. The selection of the antigen-binding domain will depend on the particular the of cancer to be treated.

Tumor antigens are proteins that are produced by tumor cells that elicit an immune response, particularly T-cell mediated immune responses. The selection of the antigen-binding domain of the invention will depend on the particular type of cancer to be treated. Tumor antigens are well known in the art and include, but are not limited to, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RUL RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and docs not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG72, CA 19-9, CA 72-4, CAM 17.1, NuMa, K-ras, beta-Catenin, CDK4, Mum-1, p 15, p 16, 43-9F, 5T 4, 791Tgp 72, alpha-fetoprotein, beta-HCG, BCA 225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In some embodiments, the immune receptor is a T cell receptor (TCR). In some embodiments, the TCR is a wildtype TCR, a high affinity TCR, or a chimeric TCR. In some embodiments, the TCR is modified to have a higher affinity for the target cell (e.g., a cancer cell) antigen than a wildtype TCR. In some embodiments, the chimeric TCR may include chimeric domains, such as the TCR comprises a co-stimulatory signaling domain at a C terminal of at least one of the chains. In some embodiments, the TCR may include a modified chain, such as a modified alpha or beta chain. Such modifications may include, but are not limited to, N-deglycosylation, altered domain (such as an engineered variable region to target a specific antigen or increase affinity), addition of one or more disulfide bonds, entire or fragment of a chain derived from a different species, and any combination thereof.

Techniques for engineering and expressing T cell receptors include, but are not limited to, the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In some embodiments, the TCR comprises specificity to a target cell antigen. The target cell antigen may include any type of protein associated with a target cell. For example, the target cell antigen may be chosen to recognize a particular disease state of the target cell. Thus, examples of cell surface markers that may act as ligands for the antigen-binding domain of the TCR including those associated with viral, bacterial and parasitic infections, autoimmune disease and cancer cells.

In some embodiments, the immune receptor is a chimeric antigen receptor (CAR). In some embodiments, the CAR comprises an extracellular domain and an intracellular domain. In some embodiments, the extracellular domain comprises a target-specific binding domain (e.g., an antigen-binding moiety) that binds to an antigen on a target cell. Examples of cell surface markers that may act as an antigen that binds to the antigen-binding domain of the CAR include those associated with viral, bacterial and parasitic infections, autoimmune disease, alloimmune disease, and cancer cells. In some embodiments, the intracellular domain, or the cytoplasmic domain, comprises a costimulatory signaling region and an intracellular signaling region.

In some embodiments, the target-specific binding domain comprises an antigen-specific antibody or fragment thereof. The target-specific binding domain can be directed to any desired antigen. In some embodiments, the target-specific binding domain may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used or a truncated chain may be used, where all or a part of the CH1, CH2, or CH3 domains may be removed or all or part of the hinge region may be removed. Examples of antibody fragments include, but are not limited to, Fab, Fab′, F(ab′)2, and Fv fragments, linear antibodies, scFv antibodies, multispecific antibodies, VHH, VNARs, and minibodies. In some embodiments, the CAR comprises a target-specific binding domain directed to a single target antigen. In some embodiments, the CAR comprises more than one target-specific binding domain directed to a single target antigen. In some embodiments, the CAR comprises more than one target-specific binding domain directed to more than one target antigen.

In some embodiments, a CAR comprises an extracellular domain comprising an autoantigen or a fragment thereof. In some embodiments, the autoantigen binds to an autoantibody. In some embodiments, the autoantibody is expressed on a B cell. In some embodiments, the B cell is a memory B cell.

In some embodiments, the CAR further comprises a hinge region that connects the antigen-binding domain with the transmembrane domain, which, in turn, connects to the intracellular domain. In exemplary embodiments, the hinge region is capable of supporting the antigen-binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen-binding domain to have a structure to optimally recognize the specific structure and density of the target antigens on a cell such as a tumor cell. The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, For example, hinge regions include glycine polymers (G)n, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142).

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region.

In some embodiments, the CAR comprises a transmembrane domain capable of spanning the plasma membrane of a cell (e.g., an immune cell or precursor thereof). The transmembrane domain is for insertion into a cell membrane, e.g., a eukaryotic cell membrane.

In some embodiments, the transmembrane domain comprises a CD8a transmembrane domain. In some embodiments, a subject CAR comprises a CD8a transmembrane domain. In some embodiments, a subject CAR comprises a CD8a hinge domain and a CD8a transmembrane domain. In some embodiments, the transmembrane domain comprises a CD28 transmembrane domain. Tolerable variations of the transmembrane and/or hinge domain will be known to those of skill in the art, while maintaining its intended function. For example, in some embodiments a transmembrane domain or hinge domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence of the transmembrane and/or hinge domain.

The transmembrane domain may be combined with any hinge domain and/or may comprise one or more transmembrane domains described herein.

The transmembrane domains described herein, such as a transmembrane region of alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen-binding domains described herein, any of the costimulatory signaling domains or intracellular domains or cytoplasmic domains described herein, or any of the other domains described herein that may be included in the CAR.

In some embodiments, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

The intracellular domain, or the cytoplasmic domain, of a CAR is responsible for activation of at least one of the normal effector functions of the immune cell in which the CAR has been placed. Effector function of a T cell, for example, may be cytolytic activity or helper activity, including the secretion of cytokines. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Preferred examples of intracellular signaling domains for use in a CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability. T cell activation can be said to be mediated by two distinct classes of cytoplasmic signaling sequence: those that initiate antigen dependent primary activation through the TCR (primary cytoplasmic signaling sequences) and those that act in an antigen-independent manner to provide a secondary or co-stimulatory signal (secondary cytoplasmic signaling sequences). Primary cytoplasmic signaling sequences that act in a stimulatory manner may contain signaling motifs which are known as immunoreceptor tyrosine-based activation motifs or ITAMs. Examples of ITAM-containing primary cytoplasmic signaling sequences that are of particular use in the invention include those derived from TCR zeta, FcR gamma, FcR beta, CD3 gamma, CD3 delta, CD3 epsilon, CD5, CD22, CD70a, CD79b, and CD66d. In some embodiments, the cytoplasmic signaling molecule comprises a cytoplasmic signaling sequence derived from CD3 zeta. In some embodiments, the cytoplasmic domain can be designed to comprise the CD3 zeta signaling domain by itself or combined with any other desired cytoplasmic domain(s) useful in the context of a CAR. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, ICOS, CD2, CD30, CD40, PD-1, lymphocyte function-associated antigen 1 (LFA-1), CD7, LIGHT, NKG2C, B7-H3, and a ligand that specifically binds with CD83, and the like. The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker. In some embodiments, the CAR comprises a cytoplasmic signaling domain comprising a single intracellular domain of a costimulatory molecule. In some embodiments, the CAR comprises a cytoplasmic signaling domain comprising more than one intracellular domain of a costimulatory molecule. In some embodiments, the more than one intracellular domain is derived from a single costimulatory molecule. In some embodiments, the more than one intracellular domain is derived from different costimulatory molecules (e.g., a third generation CAR).

In some embodiments, the cell comprising a CAR further comprises a transgenic payload (e.g., a fourth generation CAR). In some embodiments, the transgenic payload is an inducible payload, e.g., expression of the payload is inducible. In some embodiments, the transgenic payload is a cytokine. In some embodiments, the cytokine is an interleukin. In some embodiments, the interleukin is IL-12.

In some embodiments, the CAR comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, K1R2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Gene Editing Systems

In some embodiments, the payload is a nucleic acid, a polypeptide, or a combination, encoding a gene editing system. In some embodiments, the nucleic acid editing system edits DNA. In some embodiments, the nucleic acid editing system edits RNA. In some embodiments, the nucleic acid editing system is delivered to the target cell comprising a method comprising a vector comprising a nucleic acid sequence encoding the gene editing system. In some embodiments, the nucleic acid editing system is delivered to the target cell comprising a method comprising a polypeptide comprising the gene editing system. In some embodiments, the gene editing system is delivered to the target cell comprising a method comprising a nucleic and a polypeptide.

In some embodiments, the gene editing system is a Clustered Regularly Interspaced Short Palindromic Repeats (CRISPR)/Cas editing system. CRISPR and CRISPR-associated (Cas) proteins, which provide bacteria with adaptive immunity to foreign nucleic acids, have been repurposed for use in targeted genome editing in human cells and other types of cells, as well as in animals and plants. Five types of CRISPR systems (e.g., Type I, Type II, Type III, Type IV, and Type V) have been identified.

In some embodiments, the CRISPR system is a CRISPR/Cas9 system. The CRISPR/Cas9 technology originates from type II CRISPR/Cas systems, which consist of one DNA endonuclease protein, Cas9, and two small RNAs, CRISPR RNA (crRNA) and trans-activating crRNA (tracrRNA). The small RNAs or a chimeric single guide RNA (sgRNA) bind Cas9, thus forming an RNA-guided DNA endonuclease (RGEN) complex, and cleave a specific DNA target. Chromosomal double-strand blunt-end breaks (DSBs) are then repaired via homologous recombination (HR) or non-homologous end joining (NHEJ) and produce genetic modifications.

With specific regard to CRISPR-Cas systems, a variety of reagents, methods techniques, and modules are utilized according to the present disclosure. For example, DNA breaks can be generated using a CRISPR/Cas system, e.g., a type II CRISPR/Cas system. Cas9 is an exemplary Cas enzyme used according to such methods disclosed, which catalyzes DNA cleavage.

Enzymatic action by Cas9 can generate double stranded breaks at target site sequences which hybridize to at or about 20 nucleotides of a guide sequence and that have a protospacer-adjacent motif following the 20 nucleotides of the target sequence. A vector can be operably linked to an enzyme-coding sequence encoding a CRISPR enzyme, such as a Cas protein. Non-limiting examples of Cas proteins include Cas1, Cas1B, Cas2, Cas3, Cas4, Cas5, Cas5d, Cas5t, Cas5h, Cas5a, Cash, Cas7, Cas8, Cas9 (also known as Csn1 or Csx12), Cas1O, Cmr1, Cmr3, Cmr4, Cmr5, Cmr6, Csa1, Csa2, Csa3, Csa4, Csa5, CsaX, Csb1, Csb2, Csb3, Csc1, Csc2, Csd1, Csd2, Cse1, Cse2, Cse3, Cse4, Cse5e, Csf1, Csf2, Csn1, Csn2, Csm1, Csm2, Csm3, Csm4, Csm5, Csm6, Csx17, Csx14, Csx1O, Csx16, Csx3, Csx1, Csx1S, CsO, Csf4, Cst1, Cst2, Csh1, Csh2, Csy1, Csy2, Csy3, Csy4, including homologues or modified versions thereof. For example, a CRISPR enzyme can cleave of one or both strands at or around at least about 1, 2, 3, 4, 5, 6, 7, 8, 9, 10, 15, 20, 25, 50, 100, 200, 500, or more base pairs from a predetermined (e.g., first or last) nucleotide of a specific.

The contemplated CRISPR/Cas systems can be used according to the contemplated customizations to perform site specific insertion or removals of sequence. For example, a nick on an insertion site in the genome can be made by CRISPR/CAS to facilitate the insertion of a transgene at the insertion site. Alternatively, specific genes may be removed from the DNA of the cell. In often included embodiments, the presently contemplated cell customizations include methods of modulating the expression and/or activity of one or more target nucleic acid sequences in one or more cells by introducing into the cell one or more ribonucleic acid (RNA) sequences that comprise a portion that is complementary to each of the one or more target nucleic acid sequences and comprise a binding site for a CRISPR associated (Cas) protein; (ii) a Cas nucleic acid sequence or a variant thereof that encodes the Cas protein that targets but does not cleave the target nucleic acid sequence; and (iii) an effector domain. Such customizations may also include maintaining a target cell under conditions where these RNA sequences hybridize to a portion of the target nucleic acid sequences, the Cas protein binds to the RNA sequences and the effector domain modulates the expression and/or activity of the target nucleic acid, thereby modulating the expression and/or activity of the one or more target nucleic acid sequences in the cell. Exemplary CRISPR/Cas systems, methods, reagents, devices and modules contemplated herein are described, for example, in U.S. Pat. No. 10,253,316; U.S. Patent App. Pub. Nos. 20160046961, 20160298096A1, 201662427325, 20170204407; PCT Application Pub No. W02019067910A1, each of which is incorporated herein by reference in its entirety.

In some embodiments, the gene editing system is an engineered Transcription Activator-Like Effector Nuclease (TALEN). TALENs are fusions of the FokI restriction endonuclease cleavage domain with a DNA-binding transcription activator-like effector (TALE) repeat array. TALENs can be engineered to reduce off-target cleavage activity and thus to specifically bind a target DNA sequence and can thus be used to cleave a target DNA sequence, e.g., in a genome, in vitro or in vivo. Such engineered TALENs can be used to manipulate genomes in vivo or in vitro, e.g., for gene knockout or knock-ins via induction of DNA breaks at a target genomic site for targeted gene knockout through NHEJ or targeted genomic sequence genomic sequence replacement through homology-directed repair (HDR) using an exogenous DNA template.

TALENs can be designed to cleave any desired target DNA sequence, including naturally occurring and synthetic sequences. However, the ability of TALENs to distinguish target sequences from closely related off-target sequences has not been studied in depth. Understanding this ability and the parameters affecting it is of importance for the design of TALENs having the desired level of specificity for their therapeutic use and also for choosing unique target sequences to be cleaved in order to minimize the chance of off-target cleavage. TALENs can be engineered to be active only as heterodimers through the use of obligate heterodimeric FokI variants. In this configuration, two distinct TALEN monomers are each designed to bind one target half-site and to cleave within the DNA spacer sequence between the two half-sites.

In cells, e.g., in mammalian cells, TALEN-induced double-strand breaks can result in targeted gene knockout through NHEJ or targeted genomic sequence replacement through HDR using an exogenous DNA template. TALENs have been successfully used to manipulate genomes in a variety of organisms and cell lines.

In some embodiments, the gene editing system is a zinc finger nuclease (ZFN). Zinc fingers are part of a large superfamily of protein domains that can bind to DNA. A zinc finger consists of two antiparallel β strands and an α helix. The zinc ion is crucial for the stability of this domain type—in the absence of the metal ion the domain unfolds as it is too small to have a hydrophobic core. One very well explored subset of zinc-fingers (the C₂H₂ class) comprises a pair of cysteine residues in the β strands and two histidine residues in the α helix which are responsible for binding a zinc ion. The other two classes of zinc finger proteins are the C₄ and C₆ classes. Zinc fingers are important in regulation because when interacted with DNA and zinc ion, they provide a unique structural motif for DNA-binding proteins.

The binding specificity for 3-4 base pairs of a zinc finger is conferred by a short stretch of amino acid residues in the α helix. The primary position of the amino acid residues within the α helix interacting with the DNA are at positions −1, 3 and 6 relative to the first amino acid residue of the α helix. Other amino acid positions can also influence binding specificity by assisting amino acid residues to bind a specific base or by contacting a fourth base in the opposite strand, causing target-site overlap.

Zinc finger nucleases are protein chimeras comprised of a zinc finger-based DNA-binding domain and a DNA-cleavage domain. They are able to introduce double-strand breaks at specific locations within a DNA molecule which may subsequently be used to disable a specific allele or even rewrite the code it contains.

In some embodiments, the DNA-binding domain of a ZFN may be composed of two to six zinc fingers due to their supposed modularity. Each zinc finger motif is typically considered to recognize and bind to a three-base pair sequence and as such, a protein including more zinc fingers targets a longer sequence and therefore has a greater specificity and affinity to the target site. Depending upon the required specifications of the end-product, the included zinc fingers may be selected via a parallel, sequential or bipartite technique or through an in vitro cell-based technique.

In some embodiments, the ZFN comprises a FokI nuclease domain. The non-specific nuclease domain of FokI is functionally independent of its natural DNA-binding domain and is therefore employed in the construction of ZFNs. Since the FokI nuclease domain must dimerize to accomplish a double-strand break it is necessary that a nuclease is also bound to the opposite strand by virtue of another ZFN molecule bound to its target sequence as shown in the diagram. The two target sites need not be the same, so long as the ZFNs targeting both sites are present.

In order to form a dimer, two ZFN molecules must meet with their respective recognition sites not less than 4-6 base pairs apart but also not so far apart that they may not dimerise. While one ZFN molecule binds its target sequence on one strand, another ZFN molecule binds its target sequence on the opposite strand, as shown in the diagram. The nuclease domains dimerize and each cleaves its own strand, producing a DSB. FokI can be employed as a homo- or a heterodimer. The advantage of the heterodimer is that it may reduce off target effects (Miller et al., 2007).

ZFNs can be used to disable dominant mutations in heterozygous individuals by producing DSBs in the mutant allele which will, in the absence of a homologous template, be repaired by non-homologous end-joining (NHEJ). NHEJ repairs double-strand breaks by joining the two ends together and usually produces no mutations, provided that the cut is clean and uncomplicated. In some instances however, the repair will be imperfect, resulting in deletion or insertion of base-pairs, producing frame-shift and preventing the production of the harmful protein.

In some embodiments, the gene editing system is a meganuclease. A group of naturally-occurring nucleases which recognize 15-40 base pair cleavage sites commonly found in the genomes of plants and fungi may provide a less toxic genome engineering option compared to other genome editing systems, e.g., ZFNs. Such meganucleases are frequently associated with parasitic DNA elements, such as group I self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29(18): 3757-3774). The LAGLIDADG meganucleases with a single copy of the LAGLIDADG motif (“mono-LAGLID ADG meganucleases”) form homodimers, whereas members with two copies of the LAGLIDADG motif (“di-LAGLID ADG meganucleases”) are found as monomers. Mono-LAGLID ADG meganucleases such as I-Crel, I-Ceul, and I-Msol recognize and cleave DNA sites that are palindromic or pseudo-palindromic, while di-LAGLIDADG meganucleases such as I-Scel, I-Anil, and I-Dmol generally recognize DNA sites that are non-palindromic (Stoddard (2006), Q. Rev. Biophys. 38: 49-95).

Natural meganucleases from the LAGLIDADG family have been used to effectively promote site-specific genome modification in plants, yeast, Drosophila, mammalian cells and mice, but this approach has been limited to the modification of either homologous genes that conserve the meganuclease recognition sequence (Monnat et al. (1999), Biochem. Biophys. Res. Commun. 255: 88-93) or to pre-engineered genomes into which a recognition sequence has been introduced (Rouet et al. (1994), MoI. Cell. Biol. 14: 8096-106; Chilton et al. (2003), Plant Physiol. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622).

In some embodiments, the gene editing system is a retrotransposon system. In some embodiments, the system comprises a transposon. In some embodiments, the transposon is a Piggy Bac transposon. The PiggyBac (PB) transposon is a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and efficiently integrates them into TTAA chromosomal sites. The powerful activity of the PiggyBac transposon system enables genes of interest between the two ITRs in the PB vector to be easily mobilized into target genomes.

In some embodiments, a PB contains a drug inducible promoter linked to a polynucleotide coding for a chimeric antigen receptor. In some alternatives, the inducible promoter can be linked to chemokine receptor, a marker gene, and/or a cytokine. One or more PB transposons can be employed. In some embodiments, a PB comprises an inducible promoter linked to a polynucleotide coding for a first chimeric antigen receptor, another PB comprises an inducible promoter linked to a polynucleotide coding for a second and different chimeric antigen receptor, and/or a PB comprises an inducible promoter linked to a polynucleotide coding for a chemokine receptor, a chimeric antigen receptor, and a marker gene. Each element of the constructs is separated by a nucleic acid, such as that coding for a self-cleaving T2A sequence. In some embodiments, each PB differs from one another in the chimeric antigen receptor including but not limited to the spacer length and sequence, the intracellular signaling domain, and/or the marker sequence. The PB vector can be used with a constitutive lentivirus vector coding for a transcriptional activator for the inducible promoter and constitutive vector comprising the piggyback transposase linked to a constitutive promoter.

In some embodiments, the transposon is a Tc1/mariner-like transposon. In some embodiments, the Tc1/mariner-like transposon is a Sleeping Beauty (SB) transposon. The Tc1/mariner elements are likely the most widespread transposons in nature, and can transpose in species other than their hosts, making them potential tools for functional genomics in diverse organisms, including vertebrates. However, most naturally occurring Tc1/mariner-like transposons are non-functional due to the accumulation of inactivating mutations. Although no single active element has ever been identified in vertebrates, an active Tc1-like transposon called Sleeping Beauty (SB) was recently reconstructed from pieces of defective fish elements. SB functions in a variety of vertebrate species, including human and mouse cells, and is the most active member of the Tc1/mariner family. Moreover, this element has been applied recently towards gene discovery in the mouse germline, and has been shown to promote stable in vivo delivery of therapeutic genes in somatic tissues of adult mice.

Each end of the SB transposon element contains IR/DR structure consisting of two short direct repeats (DRs) within a ˜230 by imperfect terminal inverted repeat (IR). These direct repeats (˜30 bp) serve as core-binding sites for the element-encoded transposase, and the presence of both sites within an individual IR is required for efficient transposition. In addition to the DRs, the left IR of SB contains a half binding site, termed HDR, which acts as a transpositional enhancer-like sequence. Specific binding to the DRs is mediated by an N-terminal, paired-like DNA-binding domain of the transposase. The C-terminal, catalytic domain of the transposase is responsible for all DNA cleavage and strand transfer reactions and is characterized by the presence of a conserved amino acid triad, the DDE motif. This catalytic triad is found in a large group of recombinases, including many eukaryotic and bacterial transposases, retroviral integrases and the RAG1 V(D)J recombinase involved in immunoglobulin gene rearrangements.

The mobilization of SB elements is a specialized form of DNA recombination and occurs by a cut-and-paste pathway involving a DNA intermediate. This transposition process involves five distinct stages: (i) association of the transposase with its binding sites within the transposon IRs; (ii) assembly of an active synaptic complex in which the two ends of the element are paired and held together by bound transposase subunits; (iii) transposase-mediated excision of the element from its original donor site, (iv) re-insertion of the excised element into a new target site (TA-dinucleotide); and (v) repair of the cellular DNA at both the excision and re-insertion site.

### Other Payloads

In another aspect, the payload described herein may be a small molecule, lipid, protein, or polypeptide. In some embodiments, the payload is a small molecule. Examples of small molecules include, but are not limited to, small organic molecules or compounds such as any conventional agent or drug known to those of skill in the art. Exemplary small molecules include natural products, therapeutic agents (e.g., chemotherapeutic agents, antibiotics, antivirals), amino acids, or derivatives or combinations thereof. In an embodiment, the small molecule may be therapeutically active itself or may be a prodrug, which become active upon further modification. In an embodiment, a small molecular derivative retains some or all of the therapeutic activity as compared to the unmodified agent, while in another embodiment, the small molecule is a prodrug that lacks therapeutic activity but becomes active upon further modification. In another aspect, the payload may be a lipid. In another embodiment, the payload may be a protein (e.g., an antibody, enzyme, cytokine).

### Delivery of Payloads

In some embodiments, the payload delivery vehicle associated with the cell is a lipid nanoparticle (LNP). LNPs are useful for the delivery of nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc.

In some embodiments, the LNP comprises a cationic lipid. Cationic lipids are amphiphilic molecules that generally contain a lipophilic region containing one or more hydrocarbon groups, and a hydrophilic region containing at least one positively charged polar head group. These lipids may become cationic (protonated) as the pH is lowered below the pK of the ionizable group of the lipid, but is progressively more neutral at higher pH values. Cationic lipids facilitate entry of macromolecules such as nucleic acids into the cytoplasm through the cell plasma membrane by forming a positively charged (total charge) complex with macromolecules, such as nucleic acids. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N-(N′, N′-dimethylaminoethane)-carbamoyl)cholesterol (DC-Chol); and N-(1,2-dimyristyloxyprop-3-yl)-N, N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethyl-ammonium trifluoroacetate (DOSPA) and (DOPE), from GIBCO/BRL); and TRANSFECTAM® (commercially available cationic lipids comprising dioctadecylamidoglycylcarboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA), 1,17-bis(2-octylcyclopropyl)heptadecan-9-yl-4-(dimethylamino) butanoate.

In some embodiments, the cationic lipid is an amino lipid (or a pharmaceutically acceptable salts thereof (e.g., hydrochloride salt)). Suitable amino lipids useful in the invention include those described in WO 2012/016184, incorporated herein by reference in its entirety. Representative amino lipids include 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediou (DOAP), 1,2-dilinoleyloxo-3-(2-N,N-dimethylamino)ethoxypropane (DLin-EG-DMA), and 2,2-dilinoleyl-4-dimethylaminomethyl[1,3]-dioxolane (DLin-K-DMA), and pharmaceutically acceptable salts thereof (e.g., hydrochloride salts).

In some embodiments, the payload delivery vehicle associated with the cell is an expression vector. A nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In some embodiments, the expression vector is a viral vector. In some embodiments, the viral vector is a retrovirus. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and cnv. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In some embodiments, the retrovirus is a lentiviral vector. Lentiviral vectors are known in the art, see Naldini et al. (1996) Science 272:263-7; Zufferey et al. (1998) J. Virol. 72:9873-9880; Dull et al. (1998) J. Virol. 72:8463-8471; U.S. Pat. No. 6,013,516; and U.S. Pat. No. 5,994,136, which are each incorporated herein by reference in its entirety. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Lentiviral vector systems rely on the use of a “packaging cell line.” In general, the packaging cell line is a cell line whose cells are capable of producing infectious lentiviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a lentiviral vector system into lentiviral particles.

The lentiviral particles produced generally include an RNA genome (derived from the transfer plasmid), a lipid-bilayer envelope in which the Env protein is embedded, and other accessory proteins including integrase, protease, and matrix protein. As used herein, the term “lentiviral particle” is intended to mean a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a lentiviral virion including one or more of the gag structural polypeptides p7, p24, and p17, containing a lentiviral envelope including one or more of the env encoded glycoproteins p41, p120, and p160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev. The transfer plasmids may comprise a cPPT sequence, as described in U.S. Pat. No. 8,093,042.

In some embodiments, the lentiviral particle as used herein may be one as described in U.S. Patent Applications 2021/0147871, 2022/0017920, and PCT/US 2022/013947, the disclosures of which are hereby incorporated by reference in their entireties. In other embodiments, the lentiviral particle as used herein is one descried in any one of WO 2021/146627, WO 2022/146891. In other embodiments, the payload is delivered using one of the methods described in WO 2014/011987, WO 2018/013918, WO 2017/079499, WO 2019/067425, or WO 2022/081694.

In some embodiments, the vector is an adenoviral (Ad) vector. Adenoviral vectors are vectors which are based on or derived from the genome of a virus of the family Adenoviridae. There are at least 57 serotypes of human Adenoviruses, Ad1-AD57, that form seven “species” A-G. All scrotypes are similar in general structure and the functions of most proteins, but certain unique protein functions contribute to the unique properties of the serotype and the species. In some embodiments, the adenovirus is a human adenovirus from group A, B, C, D, E, F, or G. In some embodiments, the adenovirus is a human adenovirus from group B or C or D. In some embodiments, the adenoviral vector comprises a plurality of adenoviral early genes. The E1A proteins are the translation products of the first gene transcription events from the adenovirus genome within the nucleus at the E1A region.

More than 400 gene therapy trials have been or are being conducted with human Ad vectors. Most of these trials are for treatment of cancer, although some are for use of Ad vectors as vaccines in which the vector expresses a foreign antigenic protein or for gene therapy in which the vector expresses a non-mutant protein to correct a genetic defect.

In some embodiments, the Ad vector is Ad5. In some embodiments, the Ad vector is a genetically modified version of Ad5. In some embodiments, the Ad5 vector is replication defective. In some embodiments, the Ad5 vector is replication competent. Adenoviruses typically have the following genes: E1A, E1B, E2, E3, and E4. In some embodiments, the E1A and/or E1B genes are deleted. In some embodiments, the E1A and/or E1B genes are replaced by an expression cassette. In some embodiments the Ad vector lacks the E3 genes. In some embodiments, the Ad vectors are produced in complementing cell lines (e.g., retain and express the E1A and E1B genes). In some embodiments, the complementing cell lines can be selected from one of the following: HEK293, PER.C6, or N52.E6.

In some embodiments, the Ad vector is engineered to target specific cells based on chosen surface markers. In some embodiments, the Ad vector is equipped with a targeting adapter that recognizes a surface marker on a cell. In some embodiments, the targeting adapter is an antibody. In some embodiments, the target cells secrete a therapeutic molecule. In some embodiments, the target cell expresses a therapeutic molecule on the surface of the cell.

In some embodiments, the viral vector is an adeno-associated viral vector. Adeno-associated viruses (AAVs) are members of the Parvovirus family, and is a small nonenveloped, icosahedral virus with single-stranded linear DNA genomes of 4.7 kilobases (kb) to 6 kb. AAV is assigned to the genus, Dependovirus, because the virus was discovered as a contaminant in purified adenovirus stocks. AAV's life cycle includes a latent phase at which AAV genomes, after infection, are site specifically integrated into host chromosomes and an infectious phase in which, following cither adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer.

In some embodiments, the AAV vector comprises an intact AAV capsid from a single AAV serotype. In some embodiments, the AAV vector comprises an artificial capsid which contains one or more fragments of more than one AAV serotype (e.g., a chimeric capsid). In some embodiments, the AAV vector is selected from one or more of the following: AAV1, AAV2, AAV3, AAV4, AAV5, AAV6, AAV7, AAV8, AAV9, or AAVrh74 serotype. In some embodiments, the AAV vector is a pseudotyped AAV, such as a recombinant AAV (rAAV) 1/8 or rAAV2/9. In some embodiments, the AAV vector is designed to target expression in distinct tissues (e.g., muscle and liver).

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV9 and the novel serotype of the invention, AAV8. These ITRs or other AAV components may be readily isolated using techniques available to those of skill in the art from an AAV scrotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

In some embodiments, the payload delivery vehicle associated with the cell is a vesicle. In some embodiments, the vesicle is an exosome. A central feature of exosomes is their ability to contain biologically active payload within their interior space, or lumen. It is well known that exosomes contain endogenous payload including mRNA, miRNA, DNA, proteins, carbohydrates, and lipids, but the ability to direct specific loading of desired payload is currently limited. Exosomes may be loaded by overexpressing desired payloads in a producer cell, but this loading is often of limited efficiency due to stochastic localization of the payload to cellular exosome processing centers. Alternatively, purified exosomes may be loaded ex vivo by, for example, electroporation. These methods may suffer from low efficiency or be limited to small payloads, such as siRNAs. Therefore, suitable methods for generating highly efficient and well-defined loaded exosomes are needed to better enable therapeutic use and other applications of exosome-based technologies.

In some embodiments, the vesicle is a nanovesicle. Nanovesicles comprising a payload can be produced in vitro, from animal cells grown in vitro, typically mammalian cells such as murine or human cells (e.g., producer cells). In some embodiments, the nanovesicles may comprise exosomes and other extracellular vesicles naturally released from cells, as well as artificial vesicles produced by serial extrusions of cells through micro- and/or nano-filters. In other embodiments of the invention the nanovesicles are artificial nanovesicles, which may be produced by serial extrusions of cells through micro- and/or nano-filters. It is known that extruding cells through filters disintegrates the cells, and that the cell membrane and membrane fractions reassemble in the process. The yield of vesicles produced by such a method is much higher than that of naturally produced exosomes.

Artificial nanovesicles used in embodiments of the present invention may be produced by serial extrusions of producer cells, typically mammalian cells, through micro- and/or nano-filters. The nanovesicles may be produced by a method as described in US 2012/0177574, incorporated herein by reference, see in particular paragraphs [0173]-[0197]. The thus artificially produced nanovesicles retain the membrane structure of the producer cells, such as the lipid bilayer structure and membrane proteins including topology of the cell membrane surface molecules. Furthermore, the nanovesicles retain the same cytoplasmic components as the producer cell.

A nanovesicle according to embodiments of the invention may have a size up to 500 nm, for example up to 300 nm, such as up to 250 nm or 200 nm. For example, the nanovesicles may have a size in the range of 100-200 nm. However, in some embodiments the nanovesicles may be smaller than 100 nm, for example at least 50 nm or at least 80 nm.

In some embodiments, exosomes and other vesicles, e.g., extracellular vesicles (EVs), microvesicles, naturally produced and released by cells may also contain the payload and optionally also express a targeting molecule on its surface.

Exosomes, EVs, microvesicles and artificial nanovesicles have several similarities but there are also important differences between these vesicles. Exosomes are produced through a natural process, involving inward budding of the cell membrane and are formed via multivesicular bodies. Exosomes are loaded with very specific materials, e.g., nucleic acids, which is not a random selection of cellular content.

Artificial nanovesicles, produced by serial extrusions of cells through micro- and nanofilters, do contain any protein on their surface that the cell does not have, and also contains a random selection of the cytoplasm. Therefore, overexpression of targeting surface molecule in the cell membrane of an engineered cell will result in the presence of that molecule on the artificial nanovesicles. Similarly, a molecule overexpressed within the cell, will also be present within the artificial nanovesicle in high concentrations. Most importantly, the yield of artificial nanovesicles is expected to be significantly higher than the yield of exosomes or other extracellular vesicles naturally released by a cell.

In some embodiments, the vesicle comprises a targeting moiety. In some embodiments, the vesicle is produced by direct plasma membrane budding. In some embodiments, the vesicle is one as disclosed, for instance, in PCT/US2021/037053, incorporated by reference herein.

In some embodiments, the payload delivery vehicle associated with the cell is a liposome. Liposomes, or lipid bilayer vesicles, have been used or proposed for use in a variety of applications in research, industry, and medicine, particularly for the use as carriers of diagnostic or therapeutic compounds in vivo. See, for example: Lasic, D. Liposomes: from physics to applications. Elsevier, Amsterdam, 1993. Lasic, D, and Papahadjopoulos, D., eds. Medical Applications of Liposomes. Elsevier, Amsterdam, 1998. Liposomes arc usually characterized by having an interior space sequestered from an outer medium by a membrane of one or more bilayers forming a microscopic sack, or vesicle. Bilayer membranes of liposomes are typically formed by lipids, i.e., amphiphilic molecules of synthetic or natural origin that comprise spatially separated hydrophilic and hydrophobic domains. See Lasic D., 1993, supra. Bilayer membranes of the liposomes can be also formed by amphiphilic polymers and surfactants (polymerosomes, niosomes). A liposome typically serves as a carrier of an entity such as, without limitation, a chemical compound, a combination of compounds, a supramolecular complex of a synthetic or natural origin, a genetic material, a living organism, a portion thereof, or a derivative thereof, that is capable of having a useful property or exerting a useful activity. For this purpose, the liposomes are prepared to contain the desired entity in a liposome-incorporated form. The process of incorporation of a desired entity into a liposome is often referred to as “loading”. The liposome-incorporated entity may be completely or partially located in the interior space of the liposome, within the bilayer membrane of the liposome, or associated with the exterior surface of the liposome membrane. The incorporation of entities into liposomes is also referred to as encapsulation or entrapment, and these three terms are used herein interchangeably with the same meaning. The intent of the liposomal encapsulation of an entity is often to protect the entity from the destructive environment while providing the opportunity for the encapsulated entity to exert its activity mostly at the site or in the environment where such activity is advantageous but less so in other sites where such activity may be useless or undesirable. This phenomenon is referred to as delivery. For example, a drug substance within the liposome can be protected from the destruction by enzymes in the body, but become released from the liposome and provide treatment at the site of disease.

Ideally, such liposomes can be prepared to include the desired compound (i) with high loading efficiency, that is, high percent of encapsulated entity relative to the amount taken into the encapsulation process; (ii) high amount of encapsulated entity per unit of liposome bilayer material; (iii) at a high concentration of encapsulated entity, and (iv) in a stable form, i.e., with little release (leakage) of an encapsulated entity upon storage or generally before the liposome appears at the site or in the environment where the liposome-entrapped entity is expected to exert its intended activity.

### Properties of Binding

In some embodiments, the payload (e.g., or payload delivery vehicle) binds to a cell with an avidity or affinity so that the interaction between the payload (e.g., or payload delivery vehicle) and the cell is not disrupted through physical interference. In an embodiment, the physical interference is at any step after the binding step and prior to infusion back into the patient. In some embodiments, the payload comprises a binding moiety that binds to a target moiety on the surface of a target cell.

Binding of a payload (e.g., or payload delivery vehicle) and a cell may occur through any binding mechanism, including ionic interactions (e.g., cationic or anionic interactions), covalent binding, transient interactions, ligand-specific interactions, polar interactions, and others. In some embodiments, the binding moiety is an antibody or an antigen-binding fragment thereof. In some embodiments, the binding moiety is a receptor. In some embodiments, the binding moiety is a ligand of a receptor. In some embodiments, the binding moiety is a lipid. In some embodiments, the binding moiety is a carbohydrate. In some embodiments, the binding moiety is a polypeptide. In some embodiments, the binding moiety binds to a target moiety on the cell surface of the target cell.

In some embodiments, the binding moiety binds to a target moiety with an attenuated affinity. In some embodiments, binding moiety binds to a target moiety with high affinity (Kd). In some embodiments, the binding moiety binds to the target moiety with a Kd of less than 10 nM. In some embodiments, the binding moiety binds to the target moiety with a Kd of less than 9 nM. In some embodiments, the binding moiety binds to the target moiety with a Kd of less than 8 nM. In some embodiments, the binding moiety binds to the target moiety with a Kd of less than 7 nM. In some embodiments, the binding moiety binds to the target moiety with a Kd of less than 6 nM. In some embodiments, the binding moiety binds to the target moiety with a Kd of less than 5 nM. In some embodiments, the binding moiety binds to the target moiety with a Kd of less than 4 nM, 3 nM, 2.5 nM, 2 nM or 1 nM. In some embodiments, the binding moiety binds to the target moiety with a binding affinity (Kd) of about 10 nM to about 5 nM, about 5 nM to about 2 nM, about 5 nM to about 3 nM, about 5 nM to about 4 nM, about 3 nM to about 1 nM, or about 2 nM to about 1 nM.

In some embodiments, the binding of the binding moiety to the target moiety is based on pH. In some embodiments, the binding moiety binds to the target moiety at acidic pH. In some embodiments, the binding moiety binds to the target moiety at basic pH. In some embodiments, the binding moiety disassociates with the target moiety at physiological pH. In some embodiments, physiological pH is about 7. In some embodiments, physiological pH is about 7.5. In some embodiments, physiological pH is about 7 to about 7.1, about 7.1 to about 7.2, about 7.2 to about 7.3, about 7.3 to about 7.4, or about 7.4 to about 7.5. In some embodiments, physiological pH is about 7.3. In some embodiments, physiological pH is about 7.35. In some embodiments, physiological pH is about 7.4. In some embodiments, physiological pH is about 7.45.

In some embodiments, the binding moiety is a ligand of a receptor. In some embodiments, the binding moiety is a receptor which binds to a ligand. Some examples of ligand/receptor pairings include, but are not limited to, insulin/insulin receptor, low density lipoprotein/low density lipoprotein, Fc region/Fc receptor, transferrin/transferrin receptor, and GalNAc/Asialoglycoprotein receptor (ASGPR).

In some embodiments, the payload delivery vehicle comprises a cationic lipid to facilitate binding to a cell. Cationic lipids are positively charged amphiphiles consisting of three basic chemical functional domains: a hydrophilic headgroup, a hydrophilic domain, and a linker bond that tethers the cationic headgroup and a hydrophobic tail domain. The headgroups can vary from primary, secondary, and tertiary aminos, or quaternary ammonium salts as well as phosphorus, guanidino, arsenic, imidazole, and pyridinium groups. In some embodiments, the cationic lipid is DMRIE-cholesterol, DOTIM-cholesterol, EDMPC-cholesterol, DC-cholesterol, DOTMA, DOSPER, DOSPA, GL-67, bPEI, PBAE, PDMAEMA, and PEG-b-PAMA, or variants thereof.

In some embodiments, the payload delivery vehicle comprises an antibody or an antibody fragment thereof. In some embodiments, the antibody or antibody fragment thereof comprises a variable region that targets a moiety on the surface of a cell. In some embodiments, the moiety is a cell-specific marker. In some embodiments, the cell-specific marker is a marker for a PBMC. In an embodiment, the PBMC cell-specific marker is CD3. In an embodiment, the PBMC cell-specific marker is CD4. In an embodiment, the PBMC cell-specific marker is CD5. In an embodiment, the PBMC cell-specific marker is CD6. In an embodiment, the PBMC cell-specific marker is CD8. In an embodiment, the PBMC cell-specific marker is CD14. In an embodiment, the PBMC cell-specific marker is CD16. In an embodiment, the PBMC is CD19. In an embodiment, the PBMC cell-specific marker is CD20. In an embodiment, the PBMC cell-specific marker is CD34. In an embodiment, the PBMC cell-specific marker is CD56. In an embodiment, the PBMC cell-specific marker is CD117.

In some embodiments, about 1 payload is bound to a target cell. In some embodiments, more than 1 payload is bound to a target cell. In some embodiments, about 2 payloads are bound to a target cell. In some embodiments, about 3 payloads are bound to a target cell. In some embodiments, about 4 payloads are bound to a target cell. In some embodiments, about 5 payloads are bound to a target cell. In some embodiments, about 10 payloads are bound to a target cell. In some embodiments, about 20 payloads are bound to a target cell. In some embodiments, about 30 payloads are bound to a target cell. In some embodiments, about 40 payloads are bound to a target cell. In some embodiments, about 50 payloads are bound to a target cell. In some embodiments, about 100 payloads are bound to a target cell. In some embodiments, about 150 payloads are bound to a target cell.

### Diseases and Disorders

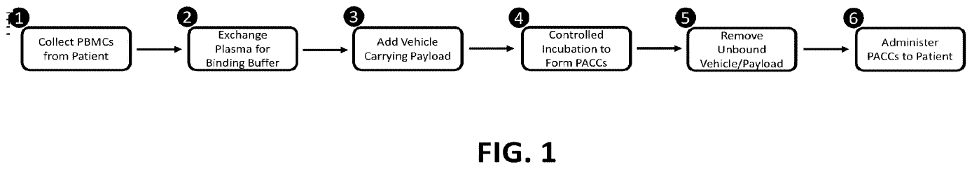

The present invention provides compositions and methods for treating various diseases and disorders. In some embodiments, the disease or disorder is treated, e.g., according to the process illustrated in FIG. 1. In some embodiments, the disease or disorder is cancer. The cancer may be, for example, a cancer of the blood, bone, bone marrow, brain, breast, diaphragm, cervix, eye, esophagus, heart, gallbladder, kidney, large intestine, liver, lung, lymph nodes, ovaries, pancreas, prostate, rectum, skin, small intestine, spleen, stomach, testes, ureter, urinary bladder, uterus, or vagina. The cancer may be a hematological malignancy, a solid tumor, a primary or a metastasizing tumor. In some embodiments, the disease or disorder is an autoimmune disease. There are two basic categories of autoimmune disease: those predominantly caused by T cells, and those predominantly caused by B cells and the autoantibodies they produce. In some embodiments, the autoimmune disease may be selected from systemic lupus erythematosus (SLE), myasthenia gravis (MG), pemphigus vulgaris (PV), coeliac disease, Crohn's disease, graft versus host disease (GvHD) (e.g., post-transplant), Grave's disease, Hashimoto's thyroiditis, multiple sclerosis, rheumatoid arthritis, Sjogren's disease, Type 1 diabetes, ulcerative colitis, and vasculitis. For example, in some embodiments, the autoimmune disease may comprise GvHD (e.g., post-transplant), Type 1 diabetes, or rheumatoid arthritis. In some embodiments, the disease or disorder is an infection, for example, a viral, bacterial, and/or a parasitic infection. For example, in some embodiments, the infection is selected from the common cold, influenza, COVID-19, gastroenteritis, hepatitis, respiratory syncytial virus (RSV), strep throat, Salmonella, tuberculosis, pertussis, Chlamydia, gonorrhea, urinary tract infections, Escherichia coli, Clostridioides difficile, giardiasis, toxoplasmosis, hookworms, and pinworms. In some embodiments, the disease or disorder is a genetic disease or disorder. Genetic diseases or disorders occur when a mutation affects a gene. For example, in some embodiments, the genetic disease or disorder is sickle cell anemia. In some embodiments, the disease or disorder is an allergic, atopic or other hypersensitivity disorder that are characterized by inappropriate or exaggerated immune reactions to foreign antigens, or alloantigens. This results in inappropriate immune reactions include those that are misdirected against intrinsic body components (self), leading to autoimmune disorders. In some embodiments, the disease or disorder is an alloimmune disease or disorder. An alloimmune disease or disorder is an immune response to non-self antigens from members of the same species, which are called alloantigens or isoantigens. For example, in some embodiments, the alloimmune disease or disorder is selected from allergic asthma, atopic dermatitis (e.g., eczema), atopy, chronic rhinosinusitis, chronic sinusitis, eosinophil-associated diseases, eosinophilic esophagitis (atopic and non-atopic), hay fever, and severe eosinophilic asthma (SEA).

### Patient Selection

In some embodiments, the subject or patient is selected for treatment comprising the method as described herein based on the presence of biomarkers. In some embodiments, the biomarkers are cancer biomarkers. In some embodiments, the cancer biomarkers comprise circulating tumor markers. In some embodiments, the cancer biomarkers comprise tumor tissue markers. In some embodiments, the cancer biomarkers may comprise one or more cancer biomarkers selected from: AFP, BCR-ABL, BRAF V600E, CA-125, CEA, HER-2, KIT, PSA, S100, CDH1, MYOD1, CDH13, p16, p14, RB1, CDKN2B, p14ARF, KRAS, p53, EGFR, TIMP1, erbB2, BRCA1, and BRCA2. In some embodiments, the biomarkers are autoimmune disease or disorder biomarkers. Exemplary autoimmune disease biomarkers include gene products of (including proteins): antibody to Heat Shock Protein 60 (anti-HSP60), Heat Shock Protein 70 (HSP 70 ), aggrecan fragments, C-propeptide of type II collagen and cartilage oligomeric matrix protein, matrix metalloprotease (MMP)-1, MMP-3 and MMP-1/inhibitor complexes thioredoxin, IL-16 and tumour necrosis factor (TNF)-alpha, neurofilament light protein and glial fibrillary acidic protein, MMP-2 and MMP-9 and TNF-alpha and soluble vascular adhesion molecule-1. In some embodiments, the biomarkers are neurological disease biomarkers. Exemplary neurological disease biomarkers include, but are not limited to, UCH-L1, GFAP, NSE, NeuN, CNPase, CAM-1, iNOS, MAP-1, MAP-2, SBDP145, SBDP120, βIII-tubulin, a synaptic protein, neuroserpin, α-internexin, LC3, neurofacin, an EAAT, DAT, nestin, cortin-1, CRMP, ICAM-1, ICAM-2, ICAM-5, VCAM-1, NCAM-1, NCAM-L1, NCAM-120, NCAM-140, NL-CAM, AL-CAM, C-CAM1, SBDP150, SBDP150i, 510013, MAP-3, MAP-4, MAP-5, MBP, Tau, NF-L, NF-M, NF-H, internexin, CB-1, CB-2; ICAM, VAM, NCAM, NL-CAM, AL-CAM, C-CAM, synaptotagmin, synaptophysin, synapsin, SNAP; CRMP-2, CRMP-1, CRMP-3, CRMP-4, iNOS, or βIII-tubulin. In some embodiments, the biomarkers are infectious disease or disorder biomarkers. Many previously identified infectious disease biomarkers associated with various infectious diseases are known to those skilled in the art and may also be found, for example, in the Infectious Disease Biomarker Database (IDBD). In some embodiments, the biomarkers are genetic disease biomarkers. Many previously identified genetic disease biomarkers associated with various genetic disease are known to those skilled in the art and may also be found, for example, in MarkerDB, an online database of molecular biomarkers (Wishart et al. (2021) MarkerDB: an online database of molecular biomarkers. Nucleic Acids Res. 2021 Jan. 8; 49(D1):D1259-D1267. doi: 10.1093/nar/gkaa1067. PMID: 33245771). In some embodiments, the biomarkers are atopic or alloimmune disease biomarkers. In some embodiments, the biomarkers are autoimmune disease biomarkers.

In some embodiments, the biomarker is a protein biomarker. In some embodiments, the protein biomarker is an antibody. In some embodiments, the protein biomarker is a cell surface protein. In some embodiments, the protein biomarker is an intracellular protein. In some embodiments, the protein biomarker is a soluble protein. In some embodiments, the protein biomarker is a secreted protein. In some embodiments, the biomarkers are nucleic acid biomarkers. In some embodiments, the nucleic acid biomarkers are DNA or RNA. In some embodiments, DNA biomarkers comprise nuclear DNA and/or mitochondrial DNA. In some embodiments, the DNA biomarker comprises a modification to the DNA (e.g., methylation, phosphorylation, acetylation). In some embodiments, the RNA biomarkers comprise messenger RNA, transfer RNA, microRNA, short hairpin RNA, ribosomal RNA, and small interfering RNA. In some embodiments, the biomarker is a sugar molecule. In some embodiments, the sugar molecule is conjugated to a protein (e.g., a glycoprotein). In some embodiments, the biomarker is a lipid. In some embodiments, the biomarker is a metabolite.

In some embodiments, the biomarker is identified and measured in a patient sample. In some embodiments, the patient sample comprises a hematological sample. In some embodiments, the hematological sample comprises red blood cells, PBMCs (e.g., T cells, B cells, NK cells, monocytes), platelets, and granulocytes (e.g., neutrophils, basophils, and eosinophils). In some embodiments, the hematological sample comprises plasma.

In some embodiments, the patient sample is a tumor sample. In some embodiments, the tumor sample is a solid tumor sample. In some embodiments, the solid tumor sample is a biopsy sample. In some embodiments, the solid tumor sample originates from a primary tumor. In some embodiments, the solid tumor sample originates from a secondary tumor or a metastases.

In some embodiments, the patient is treatment naïve. In some embodiments, the patient has been treated with one or more therapeutics. In some embodiments, the patient has been previously treated with lymphodepleting therapy (e.g., fludarabine and cyclophosphamide). In some embodiments, the patient has been previously treated with an immunotherapy (e.g., antibodies, immune cell therapy, cytokine therapy, combination therapy). In some embodiments, the patient has been previously treated with a small molecule. In some embodiments, the patient has been previously treated with radiation or radiotherapy. In some embodiments, the patient has been previously treated with a pain medication. In some embodiments, the patient has been previously treated with chemotherapy.

In some embodiments, the patient is a pediatric patient. In some embodiments, the patient is an adult. In some embodiments, the patient meets inclusion and exclusion criteria as defined by the treatment for the specific disease or disorder. In some embodiments, the patient is tested for viral, bacterial, or fungal infections prior to treatment. In some embodiments, the patient has a recurrent disease or disorder. In some embodiments, the patient has more than one disease or disorder (e.g., one or more cancers). In some embodiments, the patient has more than one disease or disorder at the same time. In some embodiments, the patient has had a previous disease or disorder. In some embodiments, the patient has an evaluable disease.

In some embodiments, the patient weighs at least 10 kilograms (kgs). In some embodiments, the patient weighs at least 40 kgs. In some embodiments, the patient weighs at least 45 kgs.

In some embodiments, the patient has a white blood cell count of at least 3,500 white blood cells per μL blood. In some embodiments, the patients has a white blood cell count of between 3,500 to 30,000 white blood cells per μL blood. In some embodiments, the patient has a white blood cell count of at least 30,000 white blood cells per μL blood. In some embodiments, the patient has a white blood cell count of between 3,500 to 50,000 white blood cells per μL blood.

In some embodiments, the patient has a circulating tumor burden below a threshold. Such a threshold indicates the level of tumor burden below which the tumors are sensitive to immunotherapy, but above which the tumors are highly resistant (Kim et al. (2021). Tumor Burden and Immunotherapy: Impact on Immune Infiltration and Therapeutic Outcomes. Front Immunol. 2021 Feb. 1; 11:629722. doi: 10.3389/fimmu.2020.629722. PMID: 33597954; PMCID: PMC7882695.). This threshold varies between cancer types and can be assessed by a physician or other clinical practitioner.

In some embodiments, the patient does not have red cell aplasia. In some embodiments, the patient does not have defects in red blood cells (e.g., reticulocytes). In some embodiments, the patient does not have reticulocytes. In some embodiments, the patient does not have red cell cold agglutinin.

### Combination Therapies

In one aspect, the PACCs as described or made by the methods described herein can be administered to a patient in combination with one or more additional therapeutics. In some embodiments, the PACCs are administered before the additional therapeutic. In some embodiments, the PACCs are administered after the additional therapeutic. In some embodiments, the PACCs are administered concurrently with the additional therapeutic. In some embodiments, the additional therapeutic is selected from a list including, but not limited to, such agents such as natalizumab, efalizumab, antiviral agents, chemotherapy, radiation, immunosuppressive agents, such as cyclosporin, azathioprine, methotrexate, mycophenolate, and FK506, antibodies, or other immuno-ablative agents such as CAMPATH, anti-CD3 antibodies, cytotoxin, fludarabine, cyclosporin, rapamycin, mycophenolic acid, steroids, FR901228, and irradiation. These drugs inhibit either the calcium dependent phosphatase calcincurin (cyclosporine and FK506) or inhibit the p70S6 kinase that is important for growth factor induced signaling (rapamycin) (Liu et al., Cell 66:807-815, 1991; Henderson et al., Immun, 73:316-321, 1991; Bterer et al., Curr. Opin. Immun. 5:763-773, 1993). In some embodiments, the additional therapeutic is a B-cell ablative therapy, such as agents that react with CD20, e.g., Rituxan. In some embodiments, the additional therapeutic is a cancer vaccine.

In some embodiments, the PACCs are obtained from a patient directly following treatment with an additional the additional therapeutic. In some embodiments, the additional therapeutic is a cancer treatment, in particular treatments with drugs that damage the immune system, shortly after treatment during the period when patients would normally be recovering from the treatment, the quality of PBMCs obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the methods as described herein, to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the additional therapeutic is an immunomodulatory agent. Non-limiting examples of immunomodulatory agents include immunostimulatory agents, checkpoint immune blockage agents, radiation therapy agents, and chemotherapy agents. In some embodiments, the immunomodulatory agent is an immunostimulatory agent. In one embodiment, the immunostimulatory agent is a cytokine, including but not limited to, IL-2, IL-3, IL-6, IL-7, IL-11, IL-12, IL-15, IL-17, and IL-21. Other exemplary immunostimulatory agents include, but are not limited to, colony stimulating factors, such as G-, M- and GM-CSF, interferons, for example, y-interferon, and the like.

In some embodiments, the additional therapeutic is a pain modulating agent. In some embodiments, the pain modulating agent is a non-steroidal anti-inflammatory drug (NSAID), such as aspirin, acetaminophen, ibuprofen, or naproxen; a triptan such as, without limitation, sumatriptan, rizatriptan, frovatriptan, zolmitriptan, eletriptan, and naratriptan; or other drug regularly used for treating pain.

In some embodiments, the additional therapeutic is an immune checkpoint blockade agent. Blockade of inhibitor immune checkpoints, such as PD-1 and CTLA-4, has been increasingly considered as an attractive strategy for cancer immunotherapy. In some embodiments, the immune checkpoint inhibitor is an inhibitor of PD-1, PD-L1, PD-L2, CTLA-4, BLTA, TIM-3, or LAG-3. In some embodiments, the immune checkpoint blockade agent is a secreted protein. In some embodiments, the immune checkpoint blockade agent is an antibody. In some embodiments, the antibody is a single chain antibody. In some embodiments, the immune checkpoint blockade agent is a single-domain antibody. In some embodiments, the immune checkpoint blockade agent is a heavy chain-only antibody. In some embodiments, the immune checkpoint blockade agent is an Fc-containing antibody (such as a full-length antibody).

In some embodiments, the additional therapeutic is a therapeutic administered to a subject or patient to address symptoms of the disease or disorder or side effects of another therapeutic agent. In some embodiments, the additional therapeutic is a pain medication. In some embodiments, the additional therapeutic is an anti-nausea therapeutic. In some embodiments, the additional therapeutic is a therapeutic administered to address disease-related cachexia. In some embodiments, the additional therapeutic is an anti-depressant. In some embodiments, the additional therapeutic is a vitamin or mineral. In some embodiments, the additional therapeutic is an anti-diarrheal medication. In some embodiments, the additional therapeutic is administered to address fatigue. In some embodiments, the additional therapeutic is administered to address a fever. In some embodiments, the additional therapeutic is administered to address nerve damage, e.g., peripheral neuropathy. In some embodiments, the additional therapeutic is administered to address chemotherapy-associated cardiovascular side effects.

In another aspect, the present disclosure comprises a method of providing a subject with a population of payload-associated cell complexes (PACCs), comprising: (i) providing a population of T cells, e.g., a population of T cells from the subject, wherein a T cell in the population of T cells comprises a binding target; (ii) extracorporeally contacting the population of T cells with a payload under conditions (e.g., time, temperature) sufficient for association of the payload with the T cell comprising the binding target, wherein the conditions are not sufficient for entry of the payload into the cell with which it is associated, thus forming a population of PACCs; and (iii) introducing the population of PACCs into the subject. In an embodiment, the population of T cells comprises CD3+ T cells, CD4+ T cells, or CD8+ T cells. In some embodiments, the population of T cells comprises CD3+ T cells. In some embodiments, a PACC of T cells described herein may be prepared by contacting the population of the subject's T cells with a lentiviral vector comprising DNA encoding (i) a chimeric antigen receptor (CAR) (e.g., a CD22 CAR) or (ii) a T cell receptor (TCR) (e.g., a TCR targeting the NY-ESO cancer antigen or HLA-A*02-presented peptide derived from PRAME). In some embodiments, a PACC of T cells described herein may be prepared by contacting the population of the subject's T cells with a lipid nanoparticle comprising an RNA encoding a CAR (e.g., a CD22 CAR). In some embodiments, a PACC of T cells described herein may be useful for delivering CAR-T therapy. In some embodiments, a PACC of T cells described herein may be useful for treating solid tumors and hematologic malignancies and genetic diseases. In some embodiments, a PACC of T cells described herein may be useful for treating melanoma or B cell non-Hodgkin's lymphoma. In an embodiment, the method comprises use of a T cell disclosed in WO2023115049A1 and EP4247963A1; WO2023193015A1, WO2023150647A1, WO2023069790A1, and WO2023019227A1; US20220267729A1, US20210317184A1, EP3516042A1, and WO2023012478A1; US20180141993A1, US20210017492A1, US20200297768A1, and WO2022147029A2; the teachings of each of which are incorporated herein by reference in its entirety. In some embodiments, the method comprises use of a lipid nanoparticle comprising an mRNA encoding a CAR, as disclosed in, e.g., US20210371494A1, WO2022261490A2, and EP4121453A2; or US20230295257A1, WO2023015221A1, and WO2022150712A1, the teachings of each of which are incorporated herein by reference in its entirety.

In another aspect, the present disclosure comprises a method of providing a subject with a population of payload-associated cell complexes (PACCs), comprising: (i) providing a population of B cells, e.g., a population of B cells from the subject, wherein a B cell in the population of B cells comprises a binding target; (ii) extracorporeally contacting the population of B cells with a payload under conditions (e.g., time, temperature) sufficient for association of the payload with the B cell comprising the binding target, wherein the conditions are not sufficient for entry of the payload into the cell with which it is associated, thus forming a population of PACCs; and (iii) introducing the population of PACCs into the subject. In an embodiment, the population of B cells comprises CD19+ B cells. In some embodiments, a PACC of B cells described herein may be prepared by contacting the population of the subject's B cells with a DNA molecule encoding an enzyme (e.g., an enzyme useful for protein replacement therapy, e.g., an enzyme useful for enzyme replacement therapy, e.g., lysosomal acid lipase (LAL) or α-L-iduronidase). In some embodiments, a PACC of B cells described herein may be useful for treating a disease or disorder that may benefit from protein replacement therapy, e.g., a lysosomal storage disorder, mucopolysaccharidosis type 1 (MPS 1), or Hurler syndrome. In an embodiment, the method comprises use of a B cell disclosed in EP4236968A1, US20180002664A1, U.S. Pat. No. 10,240,125B2, US20220193129A1, U.S. Pat. No. 11,103,582B2, US20210047619A1, US20170233452A1, and U.S. Pat. No. 9,074,223B2, the teachings of each of which is incorporated herein by reference in its entirety.

In another aspect, the present disclosure comprises a method of providing a subject with a population of payload-associated cell complexes (PACCs), comprising: (i) providing a population of monocytes, e.g., a population of monocytes from the subject, wherein a monocyte in the population of monocytes comprises a binding target; (ii) extracorporeally contacting the population of monocytes with a payload under conditions (e.g., time, temperature) sufficient for association of the payload with the monocyte comprising the binding target, wherein the conditions are not sufficient for entry of the payload into the cell with which it is associated, thus forming a population of PACCs; and (iii) introducing the population of PACCs into the subject. In an embodiment, the population of monocytes comprises CD14+ monocytes. In some embodiments, a PACC of monocytes described herein may be prepared by contacting the population of the subject's monocytes with a lipid nanoparticle comprising an RNA encoding a CAR. In some embodiments, a PACC of monocytes described herein may be primed with an antigen and, further, may be used to kill (a) cancer cells or (b) infected cells expressing the antigen. In some embodiments, a PACC of monocytes described herein may be used for treating a cancer, a genetic disease or disorder, or an infectious disease. In an embodiment, the method comprises use of a monocyte disclosed in US20230303684A1, WO2023177821A2, WO2023039221A2, U.S. Pat. Nos. 11,672,874B2, 11,628,218B2, US20230263888A1, U.S. Pat. Nos. 10,980,836B1, 11,013,764B2, WO2022226355A2, U.S. Pat. No. 11,517,589B2, WO2022251251A1, and US20220241428A1, the teachings of each of which is incorporated herein by reference in its entirety.

In another aspect, the present disclosure comprises a method of providing a subject with a population of payload-associated cell complexes (PACCs), comprising: (i) providing a population of NK cells, e.g., a population of NK cells from the subject, wherein a NK cell in the population of NK cells comprises a binding target; (ii) extracorporeally contacting the population of NK cells with a payload under conditions (e.g., time, temperature) sufficient for association of the payload with the NK cell comprising the binding target, wherein the conditions are not sufficient for entry of the payload into the cell with which it is associated, thus forming a population of PACCs; and (iii) introducing the population of PACCs into the subject. In an embodiment, the population of NK cells comprises CD16+ cells. In some embodiments, a PACC of NK cells described herein may be prepared by contacting the population of the subject's NK cells with a lentiviral vector comprising DNA encoding (i) a chimeric antigen receptor (CAR) (e.g., a NKG2D CAR). In some embodiments, a PACC of NK cells described herein may be used for treating a cancer. In an embodiment, the method comprises use of a NK cell disclosed in U.S. Pat. No. 11,154,575B2, US20210338727A1, US20220233593A1, US20220047635A1, WO2022241036A1, US20220411754A1, US20230028399A1, EP4255453A2, WO2023010018A1, and WO2023172879A2, the teachings of each of which is incorporated herein by reference in its entirety.

In another aspect, the present disclosure comprises a method of providing a subject with a population of payload-associated cell complexes (PACCs), comprising: (i) providing a population of HSCs, e.g., a population of HSCs from the subject, wherein a HSC in the population of HSCs comprises a binding target; (ii) extracorporeally contacting the population of HSCs with a payload under conditions (e.g., time, temperature) sufficient for association of the payload with the HSC comprising the binding target, wherein the conditions are not sufficient for entry of the payload into the cell with which it is associated, thus forming a population of PACCs; and (iii) introducing the population of PACCs into the subject. In an embodiment, the population of HSCs comprises CD34+ and CD59+ cells. In some embodiments, a PACC of HSCs described herein may be prepared by contacting the population of the subject's HSCs with a lentiviral vector encoding DNA molecules encoding a CRISPR-Cas9 system (e.g., a CRISPR-Cas9 system useful for treating sickle-cell anemia, e.g., a CRISPR-Cas9 system useful for correcting the mutation in the beta-globin gene). In some embodiments, a PACC of HSCs described herein may be used for treating a cancer. In an embodiment, the method comprises use of a HSC disclosed in EP3414321B8, US20220325336A1, US20220154145A1, U.S. Pat. No. 11,326,183B2, US20190390189A1, US20190184035A1, WO2019050841A1, U.S. Pat. No. 10,105,451B2, and WO2013043196A1, the teachings of each of which is incorporated herein by reference in its entirety.

### ENUMERATED EMBODIMENTS

The disclosure herein is further presented as a non-limiting list of numbered embodiments.
1. A method of providing a subject with a population of payload-associated cell complexes (PACCs), comprising:
   (i) providing a population of cells, wherein a cell in the population of cells comprises a binding target;
   (ii) extracorporeally contacting the population of cells with a payload under conditions sufficient for association of the payload with the cell comprising the binding target, wherein the conditions are not sufficient for entry of the payload into the cell with which it is associated, thus forming a population of PACCs; and
   (iii) introducing the population of PACCs into the subject,
      thereby providing subject with a population of PACCs.
2. The method of embodiment 1, wherein the population of cells in (i) comprises a population of cells from the subject.
3. The method of embodiment 1 or 2, wherein the conditions in (ii):
   wherein the conditions are sufficient for association of the payload with the cell comprising the binding target, and
   wherein the conditions are not sufficient for entry of the payload into the cell with which it is associated,
      comprise time, temperature, and/or pH.
4. The method of embodiment 3, wherein the time comprises between about 1 minute and about 180 minutes.
5. The method of embodiment 4, wherein the time comprises between about 1 minute and about 20 minutes.
6. The method of embodiment 4, wherein the time comprises between about 1 minute and about 10 minutes.
7. The method of embodiment 4, wherein the time comprises between about 5 minutes and about 30 minutes.
8. The method of embodiment 4, wherein the time comprises between about 30 minutes and about 60 minutes.
9. The method of embodiment 4, wherein the time comprises between about 60 minutes and 90 minutes.
10. The method of embodiment 4, wherein the time comprises between about 90 minutes and 120 minutes.
11. The method of embodiment 4, wherein the time comprises between about 120 minutes and 180 minutes.
12. The method of embodiment 4, wherein the time comprises 60 minutes.
13. The method of embodiment 4, wherein the time comprises 120 minutes.
14. The method of embodiment 4, wherein the time comprises 180 minutes.
15. The method of embodiment 4, wherein the time comprises less than 180 minutes.
16. The method of any one of embodiments 1-15, wherein the temperature comprises between about 0° C. to about 40° C.
17. The method of embodiment 16, wherein the temperature comprises between about 0° C. to about 10° C.
18. The method of embodiment 16, wherein the temperature comprises between about 0° C. to about 4° C.
19. The method of embodiment 16, wherein the temperature comprises between about 0° C. to about 2° C.
20. The method of embodiment 16, wherein the temperature comprises about 0° C.
21. The method of embodiment 16, wherein the temperature comprises between about 2° C. to about 4° C.
22. The method of embodiment 16, wherein the temperature comprises about 4° C.
23. The method of embodiment 16, wherein the temperature comprises between about 15° C. to about 25° C.
24. The method of embodiment 16, wherein the temperature comprises about 22° C.
25. The method of embodiment 16, wherein the temperature comprises about 25° C.
26. The method of embodiment 16, wherein the temperature comprises between about 30° C. to about 40° C.
37. The method of embodiment 16, wherein the temperature comprises about 37° C.
38. The method of embodiment 16, wherein the temperature comprises about 40° C.
39. The method of any one of embodiments 1-38, wherein the pH comprises acidic pH, physiological pH, or basic pH.
40. The method of embodiment 39, wherein the pH comprises physiological pH.
41. The method of embodiment 40, wherein the physiological pH comprises between about 7 to about 7.5
42. The method of embodiment 40 or 41, wherein the physiological pH comprises about 7.
43. The method of embodiment 40 or 41, wherein the physiological pH comprises about 7.3.
44. The method of embodiment 40 or 41, wherein the physiological pH comprises about 7.35.
45. The method of embodiment 40 or 41, wherein the physiological pH comprises about 7.4.
46. The method of embodiment 40 or 41, wherein the physiological pH comprises about 7.45.
47. The method of embodiment 40 or 41, wherein the physiological pH comprises about 7.5.
48. The method of any one of embodiments 1-47, wherein the population of PACCs comprises at least 2, 3, 4, 5, 10, 25, 50, 75, 100, 250, 500, 750, 1,000, 5,000, 7,500, 10,000, 25,000, 50,000, 75,000, 100,000, 250,000, 500,000, or more PACCs.
49. The method of embodiment 48, wherein the population of PACCs comprises at least 2 PACCs.
50. The method of embodiment 48, wherein the population of PACCs comprises at least 10 PACCs.
51. The method of embodiment 48, wherein the population of PACCs comprises at least 500 PACCs.
52. The method of embodiment 48, wherein the population of PACCs comprises at least 1,000 PACCs.
53. The method of embodiment 48, wherein the population of PACCs comprises at least 5,000 PACCs.
54. The method of embodiment 48, wherein the population of PACCs comprises at least 10,000 PACCs.
55. The method of embodiment 48, wherein the population of PACCs comprises at least 50,000 PACCs.
56. The method of embodiment 48, wherein the population of PACCs comprises at least 100,000 PACCs.
57. The method of embodiment 48, wherein the population of PACCs comprises at least 500,000 PACCs.
58. The method of embodiment 48, wherein the population of PACCs comprises more than 500,000 PACCs.
59. The method of embodiment 48, wherein the population of PACCs comprises at least 1 million PACCs.
60. The method of embodiment 48, wherein the population of PACCs comprises at least 10 million PACCs.
61. The method of embodiment 48, wherein the population of PACCs comprises at least 100 million PACCs.
62. The method of any one of embodiments 1-61, wherein the percentage of cells associated with the payload in the population of PACCs is greater than 0.5%, 1%, 2%, 10%, 20%, 30%, 40%, 50%, 60%, 70%, 80%, 90%, 95%, or more of the total cells in the population of PACCs.
63. The method of any one of embodiments 1-62, wherein the percentage of cells associated with the payload in the population of PACCs is greater than 50%, 60%, 70%, 80%, 90%, 95%, or more of the total cells in the population of PACCs.
64. The method of any one of embodiments 1-63, wherein the percentage of cells associated with the payload in the population of PACCs is greater than 50%, 60%, 70%, 80%, 90%, 95%, or more of the total target cells in the population of PACCs.
65. The method of any one of embodiments 1-64, wherein the percentage of cells associated with the payload in the population of PACCs is between 30%-90% total cells.
66. The method of embodiment 65, wherein the percentage of cells associated with the payload in the population of PACCs is between 50%-80% total cells in the population of PACCs.
67. The method of embodiment 65, wherein the percentage of cells associated with the payload in the population of PACCs is greater than 50% total cells in the population of PACCs.
68. The method of any one of embodiments 1-67, wherein the PACCs are formulated in or on a delivery vehicle.
69. The method of embodiment 68, wherein the delivery vehicle comprises a lipid nanoparticle, viral vector, vesicle, or a liposome in which the payload is disposed.
70. The method of embodiment 68 or 69, wherein the delivery vehicle comprises a lipid nanoparticle.
71. The method of embodiment 68 or 69, wherein the delivery vehicle comprises a viral vector.
72. The method of embodiment 68 or 69, wherein the delivery vehicle comprises a vesicle.
73. The method of embodiment 68 or 69, wherein the delivery vehicle comprises a liposome.
74. The method of any one of embodiments 68-73, wherein the concentration of the delivery vehicle is higher than the concentration of cells in the sample.
75. The method of any one of embodiments 68-73, wherein the concentration of the delivery vehicle is lower than the concentration of cells in the sample.
76. The method of any one of embodiments 68-75, wherein the concentration of the delivery vehicle is optimized for binding to cells in the patient sample.
77. The method of any one of embodiments 1-76, wherein the binding of the payload to the cells occurs at a selected temperature between about 0° C. to about 40° C.
78. The method of embodiment 77, wherein the binding of the payload to the cells occurs at a selected temperature between about 0° C. to about 37° C.
79. The method of embodiment 77, wherein the binding of the payload to the cells occurs at a selected temperature between about 0° C. to about 25° C.
80. The method of embodiment 77, wherein the binding of the payload to the cells occurs at a selected temperature between about 0° C. to about 22° C.
81. The method of embodiment 77, wherein the binding of the payload to the cells occurs at a selected temperature between about 0° C. to about 10° C.
82. The method of embodiment 77, wherein the binding of the payload to the cells occurs at a selected temperature between about 0° C. to about 4° C.
83. The method of embodiment 77, wherein the binding of the payload to the cells occurs at a selected temperature between about 2° C. to about 37° C.
84. The method of embodiment 77, wherein the binding of the payload to the cells occurs at a selected temperature between about 2° C. to about 25° C.
85. The method of embodiment 77, wherein the binding of the payload to the cells occurs at a selected temperature between about 2° C. to about 10° C.
86. The method of embodiment 77, wherein the binding of the payload to the cells occurs at a selected temperature between about 4° C. to about 25° C.
87. The method of embodiment 77, wherein the binding of the payload to the cells occurs at a selected temperature of about 4° C.
88. The method of embodiment 77, wherein the binding of the payload to the cells occurs at a selected temperature of about 10° C.
89. The method of embodiment 77, wherein the binding of the payload to the cells occurs at a selected temperature of about 22° C.
90. The method of embodiment 77, wherein the binding of the payload to the cells occurs at a selected temperature of about 25° C.
91. The method of embodiment 77, wherein the binding of the payload to the cells occurs at a selected temperature of about 37° C.
92. The method of embodiment 77, wherein the binding of the payload to the cells occurs at a selected temperature of about 0° C.
93. The method of embodiment 77, wherein the binding of the payload to the cells occurs at a selected temperature of about 40° C.
94. The method of any one of embodiments 1-93, wherein the payload is disposed on the surface of a cell within the PACC.
95. The method of any one of embodiments 68-94, wherein the delivery vehicle is a viral vector.
96. The method of embodiment 95, wherein the viral vector is a lentiviral vector or an adeno-associated viral (AAV) vector.
97. The method of any one of embodiments 68-94, wherein the delivery vehicle is a lipid nanoparticle.
98. The method of embodiment 97, wherein the lipid nanoparticle is a cationic lipid nanoparticle.
99. The method of any one of embodiments 68-94, wherein the delivery vehicle is a vesicle.
100. The method of embodiment 99, wherein the vesicle is an extracellular vesicle, exosome, a nanovesicle, or a microvesicle.
101. The method of embodiment 100, wherein the vesicle is an extracellular vesicle.
102. The method of embodiment 100, wherein the vesicle is an exosome.
103. The method of embodiment 100, wherein the vesicle is a nanovesicle.
104. The method of embodiment 100, wherein the vesicle is a microvesicle.
105. The method of any one of embodiments 68-104, wherein the delivery vehicle is disposed on the surface of a cell within the PACC.
106. The method of any one of embodiments 68-105, wherein the delivery vehicle is targeted to a surface molecule.
107. The method of any one of embodiments 68-106, wherein the delivery vehicle comprises a targeting moiety to a surface molecule.
108. The method of any one of embodiments 106 or 107, wherein the surface molecule is a cell-specific surface protein.
109. The method of embodiment 107, wherein the targeting moiety is an antibody.
110. The method of embodiment 109, wherein the antibody comprises CD3, CD4, CD5, CD6, CD8, CD14, CD16, or CD19.
111. The method of embodiment 110, wherein the antibody comprises CD3.
112. The method of embodiment 110, wherein the antibody comprises CD4.
113. The method of embodiment 110, wherein the antibody comprises CD5.
114. The method of embodiment 110, wherein the antibody comprises CD6.
115. The method of embodiment 110, wherein the antibody comprises CD8.
116. The method of embodiment 110, wherein the antibody comprises CD14.
117. The method of embodiment 110, wherein the antibody comprises CD16.
118. The method of embodiment 110, wherein the antibody comprises CD19.
119. The method of embodiment 110, wherein the antibody comprises CD20.
120. The method of embodiment 110, wherein the antibody comprises CD34.
121. The method of embodiment 110, wherein the antibody comprises CD56.
122. The method of embodiment 110, wherein the antibody comprises CD117.
123. The method of any one of embodiments 1-122, wherein the population of cells are selected from monocytes, macrophages, neutrophils, basophils, eosinophils, stem cells, mast cells, and dendritic cells.
124. The method of any one of embodiments 1-123, wherein the population of cells are selected from B cells, T cells, effector or regulatory T cells, hematopoietic stem cells (HSCs), natural killer cells, NK T cells, γ/δ T cells, and plasma cells.
125. The method of embodiment 124, wherein the population of cells are selected from B cells.
126. The method of embodiment 124, wherein the population of cells are selected from T cells.
127. The method of embodiment 124, wherein the population of cells are selected from HSCs.
128. The method of embodiment 124, wherein the population of cells are selected from NK cells.
129. The method of any one of embodiments 1-123, wherein the population of cells are selected from monocytes.
130. The method of any one of embodiments 1-129, wherein the payload comprises a nucleic acid, a peptide, a polypeptide, or a small molecule.
131. The method of embodiment 130, wherein the payload comprises a nucleic acid.
132. The method of embodiment 131, wherein the nucleic acid comprises DNA or RNA.
133. The method of embodiment 132, wherein the nucleic acid comprises DNA.
134. The method of embodiment 133, wherein the DNA comprises single stranded DNA.
135. The method of embodiment 133, wherein the DNA comprises double stranded DNA.
136. The method of embodiment 133, wherein the DNA comprises linear DNA.
137. The method of embodiment 133, wherein the DNA comprises circular DNA.
138. The method of embodiment 133, wherein the DNA comprises plasmid DNA or complementary DNA (cDNA).
139. The method of embodiment 138, wherein the DNA comprises plasmid DNA.
140. The method of embodiment 138, wherein the DNA comprises cDNA.
141. The method of embodiment 132, wherein the nucleic acid comprises RNA.
142. The method of embodiment 141, wherein the RNA comprises single stranded RNA.
143. The method of embodiment 141, wherein the RNA comprises double stranded RNA.
144. The method of embodiment 141, wherein the RNA comprises linear RNA.
145. The method of embodiment 141, wherein the RNA comprises circular RNA.
146. The method of embodiment 141, wherein the RNA comprises messenger RNA (mRNA), microRNA (miRNA), a miRNA inhibitor such as an antagomir or an antimir, messenger-RNA-interference complementary RNA (iRNA), multivalent RNA, Dicer substrate RNA, transfer RNA (IRNA), ribosomal RNA (rRNA), or an antisense oligonucleotides (ASO).
147. The method of embodiment 146, wherein the RNA comprises mRNA.
148. The method of embodiment 146, wherein the RNA comprises miRNA.
149. The method of embodiment 146, wherein the RNA comprises a miRNA inhibitor.
150. The method of embodiment 146, wherein the miRNA inhibitor comprises antagomirs.
151. The method of embodiment 146, wherein the miRNA inhibitor comprises antimirs.
152. The method of embodiment 146, wherein the RNA comprises iRNA.
153. The method of embodiment 146, wherein the RNA comprises multivalent RNA.
154. The method of embodiment 146, wherein the RNA comprises Dicer substrate RNA.
155. The method of embodiment 146, wherein the RNA comprises tRNA.
156. The method of embodiment 146, wherein the RNA comprises rRNA.
157. The method of embodiment 146, wherein the RNA comprises an ASO.
158. The method of any one of embodiments 131-157, wherein the nucleic comprises a naturally occurring or non-naturally occurring nucleic acid.
159. The method of any one of embodiments 131-158, wherein the nucleic acid comprises a synthetic or modified nucleic acid.
160. The method of embodiment 130, wherein the payload comprises a peptide.
161. The method of embodiment 160, wherein the peptide is cationic.
162. The method of embodiment 160, wherein the peptide is anionic.
163. The method of embodiment 160, wherein the peptide comprises at least 3 amino acids.
164. The method of embodiment 160, wherein the peptide comprises at least 5 amino acids.
165. The method of embodiment 130, wherein the payload comprises a polypeptide.
166. The method of embodiment 165, wherein the polypeptide comprises an enzyme, an antibody, or a cytokine.
167. The method of embodiment 165 or 166, wherein the polypeptide comprises an enzyme.
168. The method of embodiment 165 or 166, wherein the polypeptide comprises an antibody.
169. The method of embodiment 165 or 166, wherein the polypeptide comprises a cytokine.
170. The method of any one of embodiments 130 and 160-169, wherein the peptide or polypeptide is naturally occurring or non-naturally occurring.
171. The method of any one of embodiments 130 and 160-169, wherein the peptide or polypeptide is synthetic or modified.
172. The method of any one of embodiments 1-171, wherein the association of the payload to the cell is covalent or non-covalent.
173. The method of embodiment 172, wherein the association of the payload to the cell is covalent.
174. The method of embodiment 172, wherein the association of the payload to the cell is non-covalent.
175. The method of any one of embodiments 1-174, wherein the subject has or is diagnosed with having a disease.
176. The method of embodiment 175, wherein the disease is selected from a cancer, an autoimmune disease, an infection, a genetic disease or disorder, an allergic, atopic, or other hypersensitivity disorder, or an alloimmune disorder.
177. The method of embodiment 175 or 176, wherein the disease is selected from a cancer, an infection, or a genetic disease or disorder.
178. The method of any one of embodiments 175-177, wherein the disease is a cancer.
179. The method of embodiment 178, wherein the cancer comprises a cancer of the blood, bone, bone marrow, brain, breast, diaphragm, cervix, eye, esophagus, heart, gallbladder, kidney, large intestine, liver, lung, lymph nodes, ovaries, pancreas, prostate, rectum, skin, small intestine, spleen, stomach, testes, ureter, urinary bladder, uterus, or vagina.
180. The method of embodiment 178 or 179, wherein the cancer comprises a hematological malignancy, a solid tumor, a primary or a metastasizing tumor.
181. The method of any one of embodiments 175-177, wherein the disease is an infection.
182. The method of embodiment 181, wherein the infection is selected from a viral, bacterial, and/or a parasitic infection.
183. The method of embodiment 182, wherein the infection is a viral infection.
184. The method of embodiment 182, wherein the infection is a bacterial infection.
185. The method of embodiment 182, wherein the infection is a parasitic infection.
186. The method of any one of embodiments 181-185, wherein the infection is selected from the common cold, influenza, COVID-19, gastroenteritis, hepatitis, respiratory syncytial virus (RSV), strep throat, Salmonella, tuberculosis, pertussis, Chlamydia, gonorrhea, urinary tract infections, Escherichia coli, Clostridioides difficile, giardiasis, toxoplasmosis, hookworms, and pinworms.
187. The method of any one of embodiments 175-177, wherein the disease is a genetic disease or disorder.
188. The method of embodiment 187, wherein the genetic disease or disorder is sickle cell anemia.
189. The method of any one of embodiments 1-188, wherein the method further comprises administering to the subject an additional agent.
190. The method of embodiment 189, wherein the additional agent comprises an immune-stimulatory agent.
191. The method of any one of embodiments 1-190, wherein the introducing in (iii) is carried out by a patient-connected closed-loop device.
192. The method of any one of embodiments 1-191, wherein between 1-50% of the cells in the population comprise the binding target.
193. The method of embodiment 192, wherein between 1-40% of the cells in the population comprise the binding target.
194. The method of embodiment 192, wherein between 1-30% of the cells in the population comprise the binding target.
195. The method of embodiment 192, wherein between 1-20% of the cells in the population comprise the binding target.
196. The method of embodiment 192, wherein between 1-10% of the cells in the population comprise the binding target.
197. The method of any one of embodiments 1-191, wherein at least 50% of the cells in the population comprise the binding target.
198. The method of embodiment 197, wherein at least 60% of the cells in the population comprise the binding target.
199. The method of embodiment 197, wherein at least 70% of the cells in the population comprise the binding target.
200. The method of embodiment 197, wherein at least 80% of the cells in the population comprise the binding target.
201. The method of embodiment 197, wherein at least 90% of the cells in the population comprise the binding target.
202. The method of embodiment 197, wherein at least 95% of the cells in the population comprise the binding target.
203. The method of any one of embodiments 1-202, further comprising:
   a) connecting a parenteral inlet to the subject, wherein the parenteral inlet is adapted to parenterally receive blood from the subject;
   b) permitting the blood, or a fraction thereof, from the subject to pass through the parenteral inlet to an extracorporeal cell binding (ECCB) module configured to allow extracorporeal formation of a PACC;
   c) maintaining conditions in the ECCB module such that cells from the subject's blood and a payload form a PACC; or
   d) delivering the PACC to the subject via a parenteral outlet adapted to parenterally administer PACC to the subject.
204. The method of embodiment 203, comprising (a).
205. The method of embodiment 203, comprising (b).
206. The method of embodiment 203, comprising (c).
207. The method of embodiment 203, comprising (d).
208. The method of embodiment 203, comprising (a) and (b).
209. The method of embodiment 203, comprising (a) and (c).
210. The method of embodiment 203, comprising (a) and (d).
211. The method of embodiment 203, comprising (b) and (c).
212. The method of embodiment 203, comprising (b) and (d).
213. The method of embodiment 203, comprising (a), (b), and (c).
214. The method of embodiment 203, comprising (a), (b), and (d).
215. The method of embodiment 203, comprising (a), (c), and (d).
216. The method of embodiment 203, comprising (b), (c), and (d).
217. The method of embodiment 203, comprising (a), (b), (c), and (d).
218. The method of any one of embodiments 203-217, wherein the parental inlet, the ECCB module, and the parental outlet are in fluid connection.
219. The method of any of embodiments 203-218, wherein each of the steps (a)-(d) occurs in a closed-loop system.
220. The method of any of embodiments 1-219, wherein:
   i) a subject cell is taken from the subject,
   ii) the subject cell is contacted with a payload to form a PACC, and
   iii) the PACC introduced into the subject, and
   (i)-(iii) occur in less than 0.5, 1, 2, 4, 6, or 8 hours.
221. The method of embodiment 220, wherein (i)-(iii) occur in less than 8 hours.
222. The method of embodiment 220, wherein (i)-(iii) occur in less than 6 hours.
223. The method of embodiment 220, wherein (i)-(iii) occur in less than 4 hours.
224. The method of embodiment 220, wherein (i)-(iii) occur in less than 2 hours.
225. The method of any one of embodiments 1-224, wherein the method comprises a biphasic temperature.
226. The method of embodiment 225, wherein the first phase comprises a temperature selected from the conditions in (ii) of embodiment 1.
227. The method of embodiment 226, wherein the temperature comprises a temperature of the method of any one of embodiments 1-3, 16-38, and 77-93.
228. The method of embodiment 226, wherein the temperature comprises between about 0° C. to about 40° C.
229. The method of embodiment 225, wherein the second phase comprises a series of increases in temperature between the first phase and the subject's body temperature.
230. The method of embodiment 229, wherein the increases in temperature correspond with increments of 5° C. or 10° C.
231. A method of forming in a subject a cell that is transformed or transduced with a payload, comprising:
   introducing a population of extracorporeally formed PACCs into the subject under conditions sufficient for transformation or transfection of the cell of the PACC with the payload of the PACC in the subject; and
   allowing the transformation or transfection;
      thereby forming in a subject with a cell transformed or transfected with a payload.
232. The method of embodiment 231, wherein the transformation or transfection comprises uptake of the payload by the cell of the PACC.
233. The method of any one of embodiments 231 or 232, wherein following the transformation or transfection, the PACC comprises at least 10% transformed or transfected cells.
234. The method of embodiment 233, wherein following the transformation or transfection, the PACC comprises at least 20% transformed or transfected cells.
235. The method of embodiment 233, wherein following the transformation or transfection, the PACC comprises at least 50% transformed or transfected cells.
236. The method of embodiment 233, wherein following the transformation or transfection, the PACC comprises at least 60% transformed or transfected cells.
237. The method of embodiment 233, wherein following the transformation or transfection, the PACC comprises at least 70% transformed or transfected cells.
238. The method of embodiment 233, wherein following the transformation or transfection, the PACC comprises at least 80% transformed or transfected cells.
239. The method of embodiment 233, wherein following the transformation or transfection, the PACC comprises at least 90% transformed or transfected cells.
240. The method of any one of embodiments 231-239, wherein the transformation or transfection is confirmed by measuring the level of the payload in the subject's serum before and after the PACCs are introduced into the subject.
241. The method of embodiment 240, wherein the level of the payload is determined by mRNA or protein quantification.
242. The method of any one of embodiments 231-241, wherein the level of the payload is measured 5 minutes, 30 minutes, 1 hour, 2 hours, 12 hours, 24 hours, 48 hours, 72 hours, 1 week, 2 weeks, 1 month, 2 months, 6 month, or 1 year after the PACCs are introduced into the subject.
243. The method of any one of embodiments 231-242, further comprises acquiring a measurement of a marker in the blood (e.g., serum) of the subject, e.g., after administration.
244. A patient-connected closed-loop device for use with any one of the method of embodiments 1-243.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Example 1: Lipid Nanoparticle (LNP) Binding to Peripheral Blood Mononuclear Cells (PBMCs)

This example investigates the optimal conditions for binding and uptake of LNPs by immune cells.

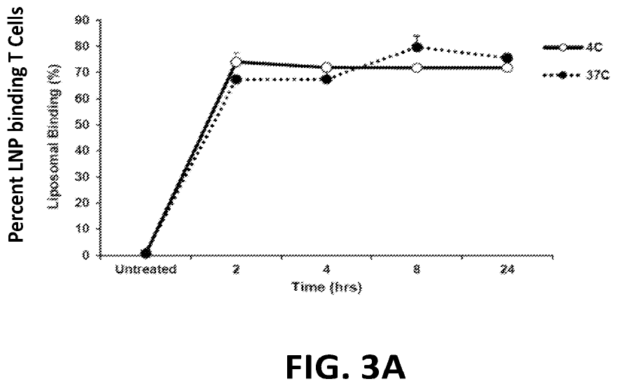

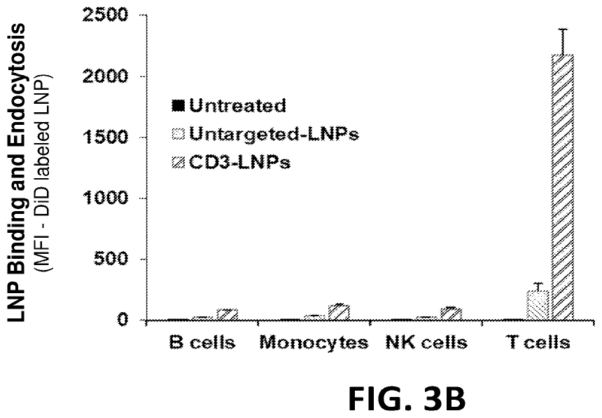

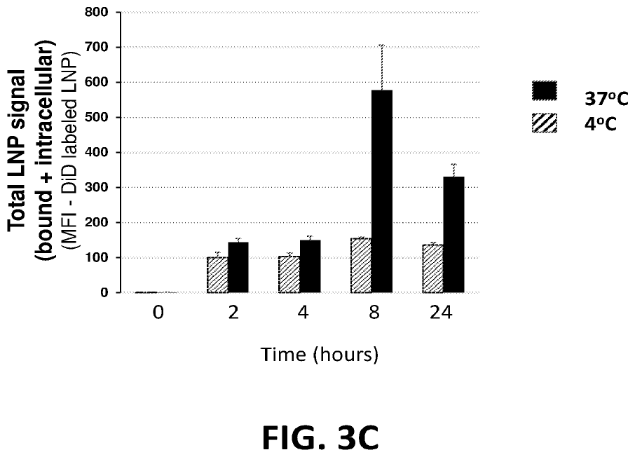

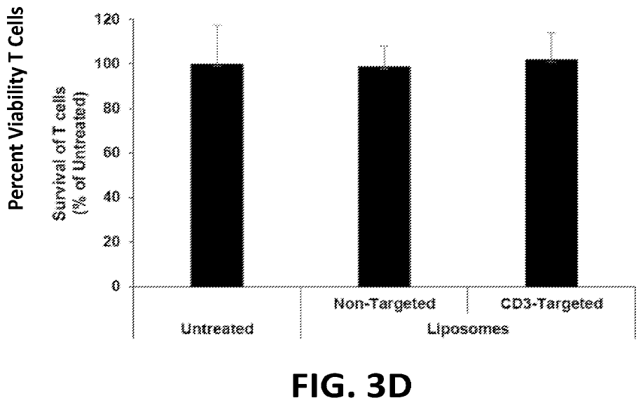

In order to determine binding conditions of LNPs, small-scale cultures of PBMCs were incubated with said LNPs loaded with DiIC 18(5); 1,1′-dioctadecyl-3,3,3′,3′-tetramethylindodicarbocyanine, 4-chlorobenzenesulfonate salt (DiD) for 1 hour at room temperature with gentle rocking. The cells were then incubated for 24 hours and were sampled at 2, 4, 8, and 24 hours. Maximal binding at both 4° C. and 37° C. occurred in less than two hours with similar kinetics (FIG. 3A).

Next, LNPs formulated with anti-CD3 antibody were used to determine if LNPs could be targeted to a specific subtype of PBMC, for example, T cells. Various types of PBMCs (B cells, monocytes, NK cells, and T cells) were incubated with the following: DiD-loaded untargeted LNPs(formulated without anti-CD3 antibody), DiD-loaded LNPs with anti-CD3 antibody (CD3-LNPs), or were left untreated. The PBMCs were incubated at 4° C. for 2 hours with the LNPs or were left untreated. The cells were then washed and incubated at 37° C. for 24 hours. After the 24 hours, the cells were stained with antibodies to differentiate PBMC subtypes (anti-CD19 for B cells; CD14 for monocytes, CD16 for NK cells, and CD3/CD4 or CD3/CD8 for T cells) and were analyzed using flow cytometry. The data was normalized to untreated cells. FIG. 3B shows that CD3-LNPs were preferentially taken up by T cells compared to other lineages within the PBMC population. Moreover, negligible binding of the untargeted LNPs was observed in all cell subtypes, demonstrating binding was indeed mediated by anti-CD3 binding to the CD3 protein found on T cells.

To investigate uptake of LNPs by T cells, T cells were incubated with CD3-targeted liposomes at either 4° C. or 37° C. to determine the rate and the timing of LNP endocytosis into the cell. Samples were taken at 0, 2, 4, 8, and 24 hours after incubation. Intracellular DiD signal peaked at 8 hours at 37° C. while those incubated at 4° C. did not show a change in intracellular signal after 2 hours (FIG. 3C).

Next, cell viability was measured in order to determine if incubation with and binding of the LNPs was detrimental to the T cells. Cell viability was measured by flow cytometry. Cells incubated with either non-targeted LNPs or CD3-targeted LNPs exhibited the same high viability as the untreated T cells (FIG. 3D).

### Example 2. Cationic-Charged LNPs Increase Binding to T Cells

This example demonstrates the binding of cationic LNPs to T cells.

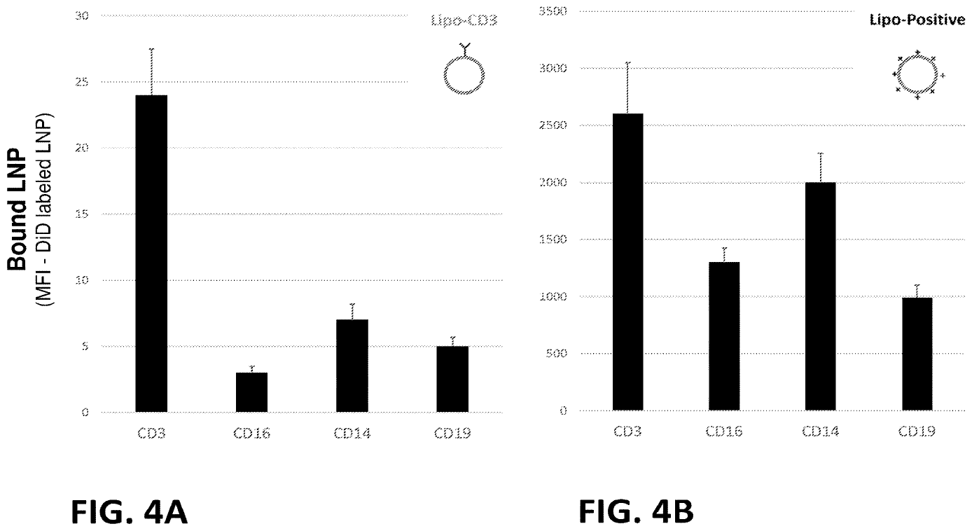

To investigate the binding of cationic LNPs to PBMCs, cells were incubated with either CD3-LNPs, which specifically target T cells, or LNPs containing cationic lipids and did not comprise anti-CD3 antibodies. Both the CD3-LNPs and the cationic LNPs were loaded with DiD in order to detect binding of the LNPs to various PBMC subtypes using FACS. Like before, CD3-LNPs specifically bound to T cells over the other PBMC subtypes (CD19-B cells; CD14--monocytes; CD16-NK cells). The cationic LNPs increased binding over all PBMC subtypes (FIGS. 4A-4B). These results show that cationic LNPs can increase binding and negate antibody specificity.

### Example 3. The Effects of Pegylated LNPs on Binding to T Cells

This example investigates the effects of LNP composition and concentration on the binding to T cells.

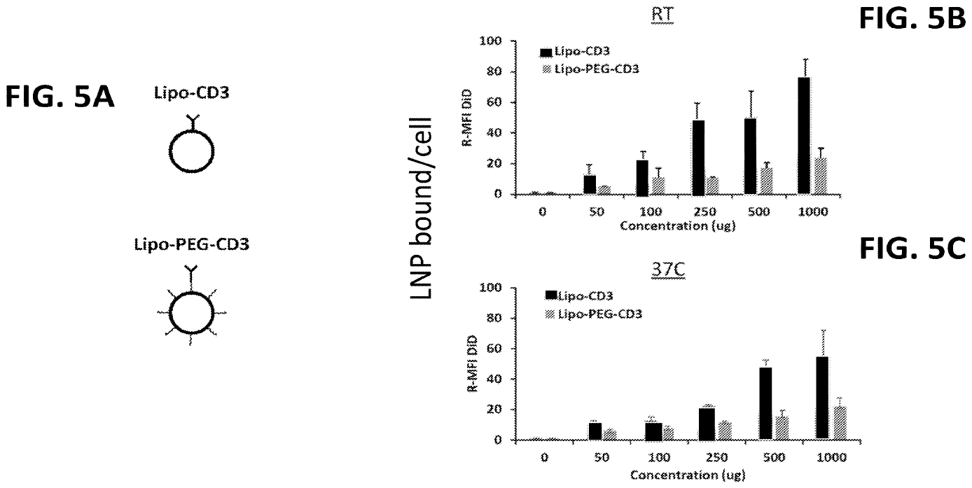

LNPs were formulated with either anti-CD3 antibody only to specifically target T cells or with anti-CD3 antibody and polyethylene glycol (PEG). The LNPs were loaded with DiD in order to measure binding via FACS. T cells were incubated with either of the LNP formulations at varying concentrations of 0, 50, 100, 250, 500, or 1000 ug. The cell-LNP mixture was incubated at either room temperature or at 37° C. for two hours. As seen in FIGS. 5A-5C, binding of LNPs was dose-dependent, with higher binding at the higher concentrations of LNP, with or without anti-CD3 antibody. However, LNPs formulated with only the anti-CD3 antibody exhibited better binding at either room temperature or 37° C., while PEGylation lowered LNP binding to T cells.

### Example 4. Polymeric Nanoparticles Bind to T Cells and are Endocytosed at 37° C.

This example investigates the binding and uptake of polymeric nanoparticles by T cells.

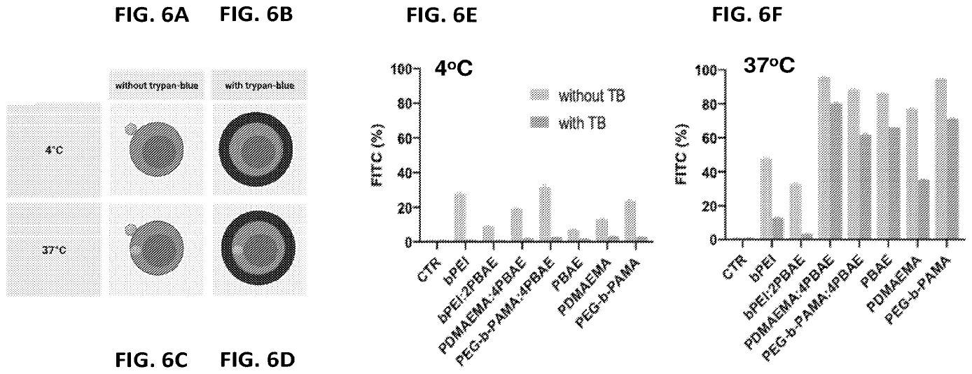

Jurkat cells were incubated for 4 hours at either 4° C. or at 37° C. with polymeric nanoparticles loaded with FITC. In order to distinguish between nanoparticles bound to the outside of the T cells from those that were endocytosed by the cells, Trypan blue was used to quench the signal from externally bound nanoparticles (FIGS. 6A-6D). Seven formulations of nanoparticles were used to compare binding: bPEI, bPEI: 2PBAE, PDMAEMA:4PBAE, PEG-b-PAMA:4PBAE, PBAE, PDMAEMA, and PEG-b-PAMA.

After four hours, the cells and nanoparticles incubated at 4° C. exhibited very little binding to up to about 30% binding. When the external signal was quenched using Trypan blue, most if not all of the signal was extinguished, which indicated very little uptake of the nanoparticles by the T cells (FIG. 6E). In contrast, high levels of binding were observed after 4 hours of incubation at 37° C. (FIG. 6F). After quenching with Trypan blue, high levels of internalizing were observed, particularly in cells incubated with nanoparticles formulated with PDMAEMA:4PBAE, PEG-b-PAMA:4PBAE, PBAE, PDMAEMA, and PEG-b-PAMA. Nanoparticles formulated with bPEI and bPEI:2PBAE exhibited low percentages of internalization. These results indicate that binding and internalization of a payload using polymeric nanoparticles as a delivery vehicle can be affected by the makeup of the nanoparticle.

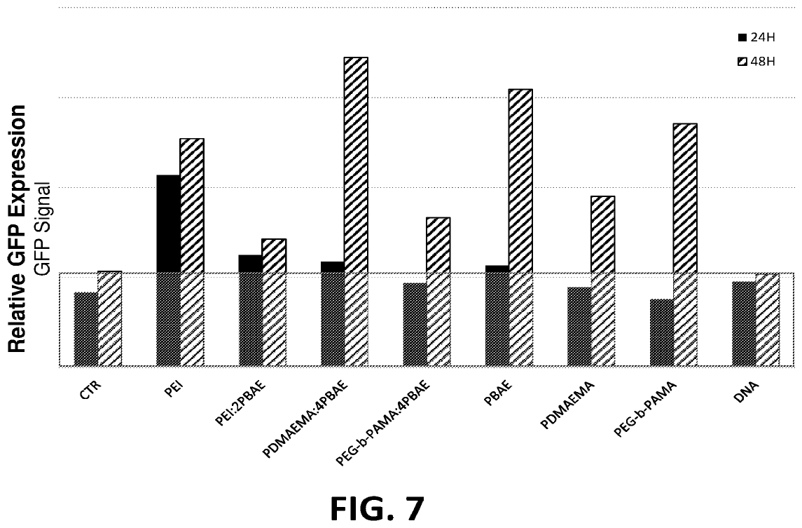

The next question was if after internalization of the polymeric nanoparticle by the target cells, would the payload be functional? The polymeric nanoparticles were used to deliver a DNA molecule encoding a green fluorescent protein (GFP) so GFP expression could be measured. GFP expression was measured 24 hours and 48 hours post-transfection. For the majority of the polymeric formulations, expression could be measured after 48 hours, with the PEI nanoparticles being the exception (FIG. 7).

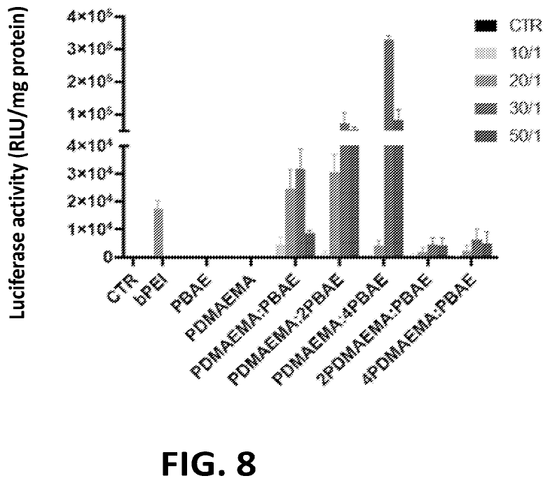

Jurkat cells were then incubated with polymeric nanoparticles at different ratios (10/1, 20/1, 30/1, or 50/1). The nanoparticles were loaded with a DNA molecule encoding the luciferase gene. Subsequent expression of the luciferase protein was measured at each ratio for each polymeric formulation. The highest levels of expression were achieved using a nanoparticle comprising PDMAEMA:4PBAE at a 30/1 ratio (FIG. 8). Significant expression was also found with nanoparticles comprising PDMAEMA:PBAE (20/1 and 30/1), PDMAEMA:2PBAE (20/1, 30/1, and 50/1), and PDMAEMA:4PBAE (50/1).

### Example 5: Characterizing LNP Binding to B Cells, Monocytes, and NK Cells

To assess whether B cells, monocytes, and NK cells could be targeted, populations of PBMCs (comprising B cells, monocytes, T cells, and NK cells) were incubated with DiD-loaded LNPs that were formulated as follows: DiD-loaded untargeted LNPs (formulated without anti-CD19, anti-CD14, or anti-CD16 antibody), DiD-loaded LNPs with anti-CD19 antibody (CD19-LNPs), DiD-loaded LNPs with anti-CD14 antibody (CD14-LNPs), DiD-loaded LNPs with anti-CD16 antibody (CD16-LNPs). As a control, one population of PBMCs (comprising B cells, monocytes, T cells, and NK cells) was left untreated. The PBMCs were incubated at 4° C. for 2 hours with the LNPs or were left untreated. The cells were then washed and incubated at 37° C. for 24 hours. Afterwards, the cells were stained with antibodies to differentiate PBMC subtypes (anti-CD19 for B cells; CD14 for monocytes, CD16 for NK cells, and CD3/CD4 or CD3/CD8 for T cells) and were analyzed using flow cytometry. The data was normalized to untreated cells. Compared to other lineages within the PBMC population, CD19-LNPs were preferentially taken up by B cells, CD14-LNPs were preferentially taken up by monocytes, and CD16-LNPs were preferentially taken up by NK cells. Negligible binding of the untargeted LNPs was observed in all cell subtypes, demonstrating binding was indeed mediated by anti-CD19 binding to the CD19 protein found on B cells, anti-CD14 binding to the CD14 protein found on monocytes, and anti-CD16 binding to the CD16 protein found on NK cells.

To investigate uptake of LNPs by B cells, monocytes, and NK cells, populations of each cell type were incubated with targeted liposomes (e.g., CD19-LNPs for B cells, CD14-LNPs for monocytes, and CD 16-LNPs for NK cells) at either 4° C. or 37° C. to determine the rate and the timing of LNP endocytosis into the cell. Samples were taken at 0, 2, 4, 8, and 24 hours after incubation. Intracellular DiD signal peaked at 8 hours at 37° C. while those incubated at 4° C. did not show a change in intracellular signal after 2 hours.

Finally, cell viability was measured in order to determine if incubation with and binding of the LNPs was detrimental to the B cells, monocytes, and NK cells. Cell viability was measured by flow cytometry. B cells, monocytes, and NK cells incubated with either (1) non-targeted LNPs or (2) CD19-, CD14-, or CD16-targeted LNPs, respectively, exhibited the same high viability as the untreated B cells, monocytes, and NK cells.

### Binding of Cationic LNPs to B Cells, Monocytes, and NK Cells

To investigate the binding of cationic LNPs to various PBMCs, B cells, monocytes, and NK cells were incubated with (a) targeted LNPs (CD19-LNPs for B cells, CD14-LNPs for monocytes, or CD16-LNPs for NK cells) or (b) untargeted LNPs (comprising no antibody) containing cationic lipids. Both the targeted LNPs and the cationic LNPs were loaded with DiD in order to detect binding of the LNPs to various PBMC subtypes using FACS. Like before, the targeted LNPs specifically bound to their target cells over the other PBMC subtypes (CD19-B cells; CD14-monocytes; CD16-NK cells). The cationic LNPs increased binding over all PBMC subtypes. These results show that cationic LNPs can increase binding and negate antibody specificity.

### The Effects of LNP Composition and Concentration on the Binding to B Cells, Monocytes, and NK Cells

LNPs were formulated with:
(a) no antibody,
(b) anti-CD19 antibody (B cells),
(c) anti-CD19 antibody and PEG,
(d) anti-CD14 antibody (monocyte cells),
(e) anti-CD14 antibody and PEG,
(f) anti-CD16 antibody (NK cells), or
(g) anti-CD16 antibody and PEG.

The LNPs (at varying concentrations of 0, 50, 100, 250, 500, or 1000 ug) were loaded with DiD in order to measure binding via FACS. B cells were incubated with (a), (b), or (c); monocyte cells were incubated with (a), (d), or (e); and NK cells were incubated with (a), (f), or (g). The cell-LNP mixture was incubated at either room temperature or at 37° C. for two hours. Binding of LNPs was dose-dependent, with higher binding at the higher concentrations of LNP, with or without the antibody (e.g., anti-CD19 antibody, anti-CD14 antibody, or anti-CD16 antibody). However, LNPs formulated with only the antibody (e.g., (b), (d), and (f)) exhibited better binding at either room temperature or 37° C., while PEGylation lowered LNP binding to B cells, monocytes, and NK cells.

### Investigating the Binding and Uptake of Polymeric Nanoparticles by B Cells, Monocytes, and NK Cells

B cells, monocytes, and NK cells were each incubated for 4 hours at either 4° C. or at 37° C. with polymeric nanoparticles loaded with FITC. In order to distinguish between nanoparticles bound to the outside of the cells from those that were endocytosed by the cells, Trypan blue was used to quench the signal from externally bound nanoparticles. Seven formulations of nanoparticles were used to compare binding: bPEI, bPEI:2PBAE, PDMAEMA:4PBAE, PEG-b-PAMA:4PBAE, PBAE, PDMAEMA, and PEG-b-PAMA.

After four hours, the cells and nanoparticles incubated at 4° C. exhibited very little binding to up to about 30% binding. When the external signal was quenched using Trypan blue, most of the signal was extinguished, which indicated very little uptake of the nanoparticles by the cells. In contrast, high levels of binding were observed after 4 hours of incubation at 37° C. After quenching with Trypan blue, high levels of internalizing were observed, particularly in cells incubated with nanoparticles formulated with PDMAEMA:4PBAE, PEG-b-PAMA:4PBAE, PBAE, PDMAEMA, and PEG-b-PAMA. Nanoparticles formulated with bPEI and bPEI:2PBAE exhibited low percentages of internalization. These results indicate that binding and internalization of a payload using polymeric nanoparticles as a delivery vehicle can be affected by the makeup of the nanoparticle.

The next question was would the payload be functional after internalization of the polymeric nanoparticle by the target cells? The polymeric nanoparticles were used to deliver a DNA molecule encoding a green fluorescent protein (GFP) so GFP expression could be measured. GFP expression was measured 24 hours and 48 hours post-transfection. For the majority of the polymeric formulations, expression could be measured after 48 hours, with the PEI nanoparticles being the exception.

B cells, monocytes, and NK cells were then incubated with polymeric nanoparticles at different ratios (10/1, 20/1, 30/1, or 50/1). The nanoparticles were loaded with a DNA molecule encoding the luciferase gene. Subsequent expression of the luciferase protein was measured at each ratio for each polymeric formulation. The highest levels of expression were achieved using a nanoparticle comprising PDMAEMA:4PBAE at a 30/1 ratio. Significant expression was also found with nanoparticles comprising PDMAEMA: PBAE (20/1 and 30/1), PDMAEMA:2PBAE (20/1, 30/1, and 50/1), and PDMAEMA:4PBAE (50/1).

### Example 6: Treating a Rare Disease with Payload-Associated Cell Complexes (PACCs) of B Cells

This example characterizes the use of PACCs of B cells to treat indications (or symptoms of indications) that can be ameliorated or palliated by protein replacement therapy (e.g., enzyme replacement therapy).

PACCs of B cells obtained by incubating B cells with polymeric nanoparticles comprising DNA molecules encoding an enzyme (e.g., an enzyme beneficial for protein replacement therapy, e.g., lysosomal acid lipase (LAL)) were prepared and evaluated (e.g., for in vitro binding and cytotoxicity) according to the methods laid out in Example 5. PACCs were purified by washing.

The resultant PACCs were then diluted in reinfusion buffer to obtain populations of cells comprising differing amounts of PACCs (e.g., 100 PACCs, 1,000 PACCs, 10,000 PACCs, 100,000 PACCs, 1,000,000 PACCs, 10,000,000 PACCs, or 100,000,000 PACCs). Each population of cells was administered at varying concentrations (e.g., diluted in 500 μL, 1 mL, or 2.5 mL reinfusion buffer) to LAL-deficient mice. Administration occurred over the course of 2.5 hours.

Next, to assess the efficacy of PACCs of B cells in treating indications (or symptoms of indications) that can be ameliorated or palliated by protein replacement therapy (e.g., enzyme replacement therapy), PACCs of B cells were prepared according to the conditions described above and were administered to subjects weighing at least 45 kg and in need of protein replacement therapy (e.g., subjects in need of enzyme replacement therapy, e.g., LAL deficiency). Serum levels of PACCs were measured (e.g., by RNA and/or protein quantification of the encoded enzyme, e.g., LAL) immediately prior to administration, 1 week-post administration, and 1 month post-administration. Symptoms were evaluated immediately prior to administration, 1 week post-administration, 1 month post-administration, and 1 year post-administration. Upon assessment of serum levels of PACCs, symptoms, and disease biomarkers (e.g., LAL biomarkers), a physician or other clinical practitioner may recommend re-administration (e.g., if serum levels of LAL biomarkers remain constant or increase). Additional administrations may be performed as needed, e.g., as determined by a physician or other clinical practitioner, on a daily, weekly, biweekly, monthly, bimonthly, or yearly basis.

### Example 7: Treating a Cancer With PACCs of Monocytes

This example characterizes the use of PACCs of monocytes to treat a cancer (e.g., a cancer that may benefit from CAR-M therapy).

PACCs of monocytes obtained by incubating monocytes with polymeric nanoparticles comprising mRNA molecules encoding a chimeric antigen receptor (CAR) (e.g., a CAR useful for CAR-therapy, e.g., a CAR comprising (i) a transmembrane region comprising CD4, CD8, or CD28 and (ii) a costimulatory domain comprising CD27, CD28, CD137, OX40, CD30, CD40, PD-1, LFA-1, CD2, CD7, Lck, DAP10, ICOS, LIGHT, NKG2C, or B7-H3) were prepared and evaluated (e.g., for in vitro binding and cytotoxicity) according to the methods laid out in Example 5. PACCs were purified by washing.

The resultant PACCs were then diluted in reinfusion to obtain populations of cells comprising differing amounts of PACCs (e.g., 100 PACCs, 1,000 PACCs, 10,000 PACCs, 100,000 PACCs, 1,000,000 PACCs, 10,000,000 PACCs, or 100,000,000 PACCs). Each population of cells was administered at varying concentrations (e.g., diluted in 500 μL, 1 mL, or 2.5 mL reinfusion buffer) to mice (e.g., Eμ-Myc or MMTV-Myc transgenic mice). Administration occurred over the course of 2.5 hours.

Next, to assess the efficacy of PACCs of monocytes in treating cancer, PACCs of monocytes were prepared according to the conditions described above and were administered to subjects having a cancer and weighing at least 45 kg. Serum levels of PACCs (e.g., by RNA and/or protein quantification of the encoded CAR) were measured immediately prior to administration, 1 week-post administration, and 1 month post-administration. Symptoms were evaluated immediately prior to administration, 1 week post-administration, 1 month post-administration, and 1 year post-administration. Upon assessment of serum levels of PACCs, symptoms, and disease biomarkers (e.g., cancer biomarkers), a physician or other clinical practitioner may recommend re-administration (e.g., if serum levels of the cancer biomarkers remain constant or increase). Additional administrations may be performed as needed, e.g., as determined by a physician or other clinical practitioner, on a daily, weekly, biweekly, monthly, bimonthly, or yearly basis.

### Example 8: Treating a Cancer With PACCs of T Cells (Method A)

This example characterizes the use of PACCs of T cells to treat a cancer (e.g., melanoma).

PACCs of T cells obtained by incubating T cells with polymeric nanoparticles comprising a lentiviral vector encoding DNA molecules encoding a T cell receptor (TCR) (e.g., a TCR useful for TCR-T therapy, e.g., a TCR capable of binding an antigenic peptide, e.g., an antigen peptide derived from MAGE-2) were prepared and evaluated (e.g., for in vitro binding and cytotoxicity) according to the methods laid out in Examples 1-4. PACCs were purified by washing.

The resultant PACCs were then diluted in reinfusion buffer to obtain populations of cells comprising differing amounts of PACCs (e.g., 100 PACCs, 1,000 PACCs, 10,000 PACCs, 100,000 PACCs, 1,000,000 PACCs, 10,000,000 PACCs, or 100,000,000 PACCs). Each population of cells was administered at varying concentrations (e.g., diluted in 500 μL, 1 mL, or 2.5 mL reinfusion buffer) to mice (e.g., C57BL/6J mice chemically induced with melanoma).

Next, to assess the efficacy of PACCs of T cells in treating cancer, PACCs of T cells were prepared according to the conditions described above and were administered to subjects having a cancer (e.g., a melanoma) and weighing at least 45 kg. Serum levels of PACCs (e.g., by RNA and/or protein quantification of the encoded TCR) were measured immediately prior to administration, 1 week-post administration, and 1 month post-administration. Symptoms were evaluated immediately prior to administration, 1 week post-administration, 1 month post-administration, and 1 year post-administration. Upon assessment of serum levels of PACCs, symptoms, and disease biomarkers (e.g., cancer biomarkers), a physician or other clinical practitioner may recommend re-administration (e.g., if serum levels of the cancer biomarkers remain constant or increase). Additional administrations may be performed as needed, e.g., as determined by a physician or other clinical practitioner, on a daily, weekly, biweekly, monthly, bimonthly, or yearly basis.

### Example 9: Treating a Cancer With PACCs of T Cells (Method B)

This example characterizes the use of PACCs of monocytes to treat a cancer (e.g., a cancer that may benefit from CAR-T therapy, e.g., acute lymphoblastic leukemia (ALL)).

PACCs of T cells obtained by incubating T cells with polymeric nanoparticles comprising mRNA molecules encoding a CAR (e.g., a CAR useful for CAR-therapy, e.g., a CAR comprising a CD22-specific antigen-binding domain) were prepared and evaluated (e.g., for in vitro binding and cytotoxicity) according to the methods laid out in Examples 1-4. PACCs were purified by washing.

The resultant PACCs were then diluted in reinfusion buffer to obtain populations of cells comprising differing amounts of PACCs (e.g., 100 PACCs, 1,000 PACCs, 10,000 PACCs, 100,000 PACCs, 1,000,000 PACCs, 10,000,000 PACCs, or 100,000,000 PACCs). Each population of cells was administered at varying concentrations (e.g., diluted in 500 μL, 1 mL, or 2.5 mL reinfusion buffer) to mice (e.g., ALL mice, such as immunodeficient mice implanted with patient-derived CD 22 BALL tumor cells). Administration occurred over the course of 2.5 hours.

Next, to assess the efficacy of PACCs of T cells in treating cancer, PACCs of T cells were prepared according to the conditions described above and were administered to subjects having a cancer (e.g., ALL) and weighing at least 45 kg. Serum levels of PACCs (e.g., by RNA and/or protein quantification of the encoded CAR) were measured immediately prior to administration, 1 week-post administration, and 1 month post-administration. Symptoms were evaluated immediately prior to administration, 1 week post-administration, 1 month post-administration, and 1 year post-administration. Upon assessment of serum levels of PACCs, symptoms, and disease biomarkers (e.g., cancer biomarkers), a physician or other clinical practitioner may recommend re-administration (e.g., if serum levels of the cancer biomarkers remain constant or increase). Additional administrations may be performed as needed, e.g., as determined by a physician or other clinical practitioner, on a daily, weekly, biweekly, monthly, bimonthly, or yearly basis.

### Example 10: Treating a Cancer With PACCs of NK Cells

This example characterizes the use of PACCs of NK cells to treat a cancer (e.g., non-small cell lung carcinoma (NSCLC)).

PACCs of NK cells obtained by incubating NK cells with polymeric nanoparticles comprising a lentiviral vector encoding DNA molecules encoding a CAR (e.g., a CAR useful for CAR-NK therapy, e.g., a CAR comprising CD244-specific antigen-binding domain) were prepared and evaluated (e.g., for in vitro binding and cytotoxicity) according to the methods laid out in Example 5. PACCs were purified by washing.

The resultant PACCs were then diluted in reinfusion buffer to obtain populations of cells comprising differing amounts of PACCs (e.g., 100 PACCs, 1,000 PACCs, 10,000 PACCs, 100,000 PACCs, 1,000,000 PACCs, 10,000,000 PACCs, or 100,000,000 PACCs). Each population of cells was administered at varying concentrations (e.g., diluted in 500 μL, 1 mL, or 2.5 mL reinfusion buffer) to mice (e.g., FVB mice). Administration occurred over the course of 2.5 hours.

Next, to assess the efficacy of PACCs of NK cells in treating cancer, PACCs of NK cells were prepared according to the conditions described above and were administered to subjects having a cancer (e.g., NSCLC) and weighing at least 45 kg. Serum levels of PACCs (e.g., by RNA and/or protein quantification of the encoded CAR) were measured immediately prior to administration, 1 week-post administration, and 1 month post-administration. Symptoms were evaluated immediately prior to administration, 1 week post-administration, 1 month post-administration, and 1 year post-administration. Upon assessment of serum levels of PACCs, symptoms, and disease biomarkers (e.g., NSCLC biomarkers, e.g., TP53 and KRAS), a physician or other clinical practitioner may recommend re-administration (e.g., if serum levels of the NSCLC biomarkers remain constant or increase). Additional administrations may be performed as needed, e.g., as determined by a physician or other clinical practitioner, on a daily, weekly, biweekly, monthly, bimonthly, or yearly basis.

### Example 11. Treating Sickle Cell Anemia With PACCs of Hematopoietic Stem Cells (HSCs)

This example characterizes the use of PACCs of HSCs to treat a genetic disorder (e.g., sickle cell anemia).

PACCs of HSCs obtained by incubating HSCs with polymeric nanoparticles comprising a lentiviral vector encoding DNA molecules encoding a CRISPR-Cas9 system (e.g., a CRISPR-Cas9 system useful for treating sickle-cell anemia, e.g., a CRISPR-Cas9 system useful for correcting the mutation in the beta-globin gene) were prepared and evaluated (e.g., for in vitro binding and cytotoxicity) according to the methods laid out in Example 5. PACCs were purified by washing.

The resultant PACCs were then diluted in reinfusion buffer to obtain populations of cells comprising differing amounts of PACCs (e.g., 100 PACCs, 1,000 PACCs, 10,000 PACCs, 100,000 PACCs, 1,000,000 PACCs, 10,000,000 PACCs, or 100,000,000 PACCs). Each population of cells was administered at varying concentrations (e.g., diluted in 500 μL, 1 mL, or 2.5 mL reinfusion buffer) to mice (e.g., homozygous “sickle” mice obtained, e.g., as in Javazon et al. (2012) Hematopoietic stem cell function in a murine model of sickle cell disease. Anemia. 2012; 2012:387385. doi: 10.1155/2012/387385. Epub 2012 Jun. 4. PMID: 22701784; PMCID: PMC3372279.). Administration occurred over the course of 2.5 hours.

Next, to assess the efficacy of PACCs of HSCs in treating sickle cell anemia, PACCs of HSCs were prepared according to the conditions described above and were administered to subjects having a sickle cell anemia and weighing at least 45 kg. Serum levels of PACCs (e.g., by RNA and/or protein quantification of the encoded CAR) were measured immediately prior to administration, 1 week-post administration, and 1 month post-administration. Symptoms were evaluated immediately prior to administration, 1 week post-administration, 1 month post-administration, and 1 year post-administration. Upon assessment of serum levels of PACCs, symptoms, and clinical testing or monitoring of sickle cell anemia (e.g., peripheral blood smear, solubility and sickling, capillary electrophoresis, and/or isoelectric focusing), a physician or other clinical practitioner may recommend re-administration (e.g., if the patient's condition remains constant or worsens). Additional administrations may be performed as needed, e.g., as determined by a physician or other clinical practitioner, on a daily, weekly, biweekly, monthly, bimonthly, or yearly basis.

1. A method of providing a subject with a population of payload-associated cell complexes (PACCs), comprising:
(i) providing a population of cells, e.g., a population of cells from the subject, wherein a cell in the population of cells comprises a binding target;
(ii) extracorporeally contacting the population of cells with a payload under conditions sufficient for association of the payload with the cell comprising the binding target, wherein the conditions are not sufficient for entry of the payload into the cell with which it is associated, thus forming a population of PACCs; and
(iii) introducing the population of PACCs into the subject,
thereby providing subject with a population of PACCs.

2. The method of any of claim 1, wherein the population of PACCs comprises at least 2, 3, 4, 5, 10, 25, 50, 75, 100, 250, 500, 750, 1,000, 5,000, 7,500, 10,000, 25,000, 50,000, 75,000, 100,000, 250,000, 500,000, or more PACCs.

3. The method of claim 2, wherein the population of PACCs comprises at least 100,000 or more PACCs.

4. The method of claim 2, wherein the population of PACCs comprises at least 1 million or more PACCs.

5. The method of claim 2, wherein the population of PACCs comprises at least 10 million or more PACCs.

6. The method of claim 2, wherein the population of PACCs comprises at least 100 million or more PACCs.

7. The method of claim 1, wherein the percentage of cells associated with the payload in the population of PACCs is greater than 0.5%, 1%, 2%, 10%, 20%, 30%, 40%, 50%, 60%, 70%, 80%, 90%, 95%, or more of the total cells in the population of PACCs.

8. The method of claim 7, wherein the percentage of cells associated with the payload in the population of PACCs is greater than 50%, 60%, 70%, 80%, 90%, 95%, or more of the total cells in the population of PACCs.

9. The method of claim 1, wherein the percentage of cells associated with the payload in the population of PACCs is greater than 50%, 60%, 70%, 80%, 90%, 95%, or more of the total target cells in the population of PACCs.

10. The method of claim 1, wherein the percentage of cells associated with the payload in the population of PACCs is between 30%-90% total cells in the population of PACCs.

11. The method of claim 1, wherein the conditions sufficient for association of the payload with a cell within the population of PACCs comprise:
(i) contacting the cells with the payload for between about 0 to about 10 hours.

12. The method of claim 11, wherein the contacting the cells with the payload is between about 5 minutes and 30 minutes.

13. The method of claim 11, wherein the contacting the cells with the payload is between about 30 minutes and 1 hour.

14. The method of claim 11, wherein the contacting the cells with the payload is between about 1 hour and 1.5 hours.

15. The method of claim 1, wherein the PACCs are formulated in or on a delivery vehicle.

16. The method of claim 15, wherein the delivery vehicle comprises a lipid nanoparticle, viral vector, vesicle, or a liposome in which the payload is disposed.

17. The method of claim 15, wherein the concentration of the delivery vehicle is higher than the concentration of cells in the sample.

18. The method of claim 15, wherein the concentration of the delivery vehicle is lower than the concentration of cells in the sample.

19. The method of claim 15, wherein the concentration of the delivery vehicle is optimized for binding to cells in the patient sample.

20. The method of claim 15, wherein the binding of the payload to the cells occurs at a selected temperature 0 to 40 degrees Celsius.

21. The method of claim 15, wherein the payload is disposed on the surface of a cell within the PACC.

22. The method of claim 16, wherein the delivery vehicle is a viral vector.

23. The method of claim 22, wherein the viral vector is a lentiviral vector or an adeno-associated viral (AAV) vector.

24. The method of claim 16, wherein the delivery vehicle is a lipid nanoparticle.

25. The method of claim 24, wherein the lipid nanoparticle is a cationic lipid nanoparticle.

26. The method of claim 16, wherein the delivery vehicle is a vesicle.

27. The method of claim 26, wherein the vesicle is an extracellular vesicle, exosome, a nanovesicle, or a microvesicle.

28. The method of claim 15, wherein the delivery vehicle is disposed on the surface of a cell within the PACC.

29. The method of claim 28, wherein the delivery vehicle is targeted to a surface molecule.

30. The method of claim 29, wherein the delivery vehicle comprises a targeting moiety to a surface molecule.

31. The method of claim 29, wherein the surface molecule is a cell-specific surface protein.

32. The method of claim 30, wherein the targeting moiety is an antibody.

33. The method of claim 1, wherein the population of cells are selected from monocytes, macrophages, neutrophils, basophils, eosinophils, stem cells, mast cells, and dendritic cells.

34. The method of claim 1, wherein the population of cells are selected from B cells, T cells, effector or regulatory T cells, hematopoietic stem cells (HSCs), natural killer cells, NK T cells, γ/δ T cells, and plasma cells.

35. The method of claim 1, wherein the payload comprises a nucleic acid, a peptide, a polypeptide, or a small molecule.

36. The method of claim 35, wherein the nucleic acid comprises DNA or RNA.

37. The method of claim 1, wherein the association of the payload to the cell is covalent or non-covalent.

38. The method of claim 1, wherein the subject has or is diagnosed with having a disease.

39. The method of claim 1, wherein the method further comprises administering to the subject an additional agent, e.g., an immune-stimulatory agent.

40. The method of claim 1, wherein the introducing in (iii) is carried out by a patient-connected closed-loop device.

41. The method of claim 1, wherein between 1-10% of the cells in the population comprise the binding target.

42. The method of claim 1, further comprising:
a) connecting a parenteral inlet to the subject, wherein the parenteral inlet is adapted to parenterally receive blood from the subject;
b) permitting the blood, or a fraction thereof, from the subject to pass through the parenteral inlet to an extracorporeal cell binding (ECCB) module configured to allow extracorporeal formation of a PACC;
c) maintaining conditions in the ECCB module such that cells from the subject's blood and a payload form a PACC; and
d) delivering the PACC to the subject via a parenteral outlet adapted to parenterally administer PACC to the subject.

43. The method of claim 42, wherein the parental inlet, the ECCB module, and the parental outlet are in fluid connection.

44. The method of claim 43, wherein each of the steps (a)-(d) occurs in a closed-loop system.

45. The method of claim 44, wherein:
i) a subject cell is taken from the subject,
ii) the subject cell is contacted with a payload to form a PACC, and
iii) the PACC introduced into the subject, and i-iii occur in less than 0.5, 1, 2, 4, 6, or 8 hours.

46. A method of forming in a subject a cell that is transformed or transduced with a payload, comprising:
introducing a population of extracorporeally formed PACCs into the subject under conditions sufficient for transformation or transfection of the cell of the PACC with the payload of the PACC in the subject; and
allowing the transformation or transfection;
thereby forming in a subject with a cell transformed or transfected with a payload.

47. A patient-connected closed-loop device for use with the method of claims 1.

48. A patient-connected closed-loop device for use with the method of claim 46.
